(19) United States
(12) Patent Application Publication
(10) Pub. No.: US 2023/0321317 A1
(43) Pub. Date: Oct. 12, 2023

(54) OSSEOINTEGRATIVE IMPLANTS

(71) Applicant: CTL Medical Corporation, Addison, TX (US)

(72) Inventors: Sean SUH, Milltown, NJ (US); Jon SUH, Ambler, PA (US); Danny CHON, Addison, TX (US)

(21) Appl. No.: 18/209,972

(22) Filed: Jun. 14, 2023

Related U.S. Application Data
(63) Continuation-in-part of application No. 16/799,248, filed on Feb. 24, 2020, now abandoned.
Application No. 16/799,248 is a continuation-in-part of application No. 16/726,043, filed on Dec. 23, 2019.
(60) Provisional application No. 62/809,410, filed on Feb. 22, 2019.
(60) Provisional application No. 62/783,447, filed on Dec. 21, 2018.
(60) Provisional application No. 62/783,491, filed on Dec. 21, 2018.
(60) Provisional application No. 62/795,418, filed on Jan. 22, 2019.
(60) Provisional application No. 62/809,400, filed on Feb. 22, 2019.
(60) Provisional application No. 62/812,833, filed on Mar. 1, 2019.

(51) Int. Cl. A61L 24/00; A61L 24/02

(52) U.S. Cl. CPC A61L 24/0042; A61L 24/02; A61L 24/0015; A61L 2430/38; A61L 2300/412

(57) ABSTRACT

Disclosed are devices, systems and related surgical methods including implantable devices having a plurality of osteo-inductive, osteoconductive and/or osseointegrative surface features that may be useful in joint and/or bone replacement implants used in spinal surgeries, dental surgeries and/or other orthopedic and/or general surgical procedures.

### CROSS-REFERENCE TO RELATED APPLICATIONS

This application is a continuation-in-part of U.S. patent application Ser. No. 16/799,248 entitled “SETTABLE SILICON NITRIDE CEMENTS” filed Feb. 24, 2020, which in turn claims priority to and benefit of U.S. Provisional Patent Application No. 62/809,410 entitled “SI3N4 MIXED BONE CEMENT AND RESORBABLE GRANULE” filed Feb. 22, 2019, and U.S. Provisional Patent Application No. 62/869,189 entitled “RESORBABLE BONE CEMENT GRANULES” filed Jul. 1, 2019, the disclosures of which are each incorporated by reference herein in their entireties.

This application further is a continuation-in-part of U.S. patent application Ser. No. 16/726,043 entitled “SILICON NITRIDE IMPLANTS AND COATINGS” filed Dec. 23, 2019, which in turn claims priority to and benefit of U.S. Provisional Patent Application No. 62/783,447 entitled “Bone Growth Enhancing Screws” filed Dec. 21, 2018; U.S. Provisional Patent Application No. 62/783,491 entitled “ANTI-ROTATION RODS” filed Dec. 21, 2018; U.S. Provisional Patent Application No. 62/795,418 entitled “BONE GRAFT BLOCK GEOMETRY” filed Jan. 22, 2019; U.S. Provisional Patent Application No. 62/809,400 entitled “POROUS CAGE WITH IMPACTION INSERT” filed Feb. 22, 2019 and U.S. Provisional Patent Application No. 62/812,833 entitled “BONE GROWTH ACTIVATION IMPLANTS” filed Mar. 1, 2019. The disclosures of each of these references are incorporated by reference herein in their entireties.

### FIELD OF THE INVENTION

The present subject matter relates generally to implants and/or components thereof that incorporate a variety of surface and/or subsurface treatments to promote bone growth into and/or through the implant, which may be useful in joint and/or bone replacement implants used in spinal surgeries, dental surgeries and/or other orthopedic procedures. In various embodiments, the implants can optionally incorporate silicon nitride in various forms as a component.

### BACKGROUND OF THE INVENTION

The spinal column of vertebrates provides support to bear weight and protection to the delicate spinal cord and spinal nerves. The spinal column includes a series of vertebrae stacked on top of each other. There are typically seven cervical (neck), twelve thoracic (chest), and five lumbar (low back) segments. Each vertebra has a cylindrical shaped vertebral body in the anterior portion of the spine with an arch of bone to the posterior, which covers the neural structures. Between each vertebral body is an intervertebral disk, a cartilaginous cushion to help absorb impact and dampen compressive forces on the spine. To the posterior, the laminar arch covers the neural structures of the spinal cord and nerves for protection. At the junction of the arch and anterior vertebral body are articulations to allow movement of the spine.

Various types of problems can affect the structure and function of the spinal column. These can be based on degenerative conditions of the intervertebral disk or the articulating joints, traumatic disruption of the disk, bone or ligaments supporting the spine, tumor or infection. In addition, congenital or acquired deformities can cause abnormal angulation or slippage of the spine. Anterior slippage (spondylolisthesis) of one vertebral body on another can cause compression of the spinal cord or nerves. Patients who suffer from one of more of these conditions often experience extreme and debilitating pain and can sustain permanent neurological damage if the conditions are not treated appropriately.

Various physical conditions can manifest themselves in the form of damage or degeneration of an intervertebral disc, the result of which is mild to severe chronic back pain. Intervertebral discs serve as “shock” absorbers for the spinal column, absorbing pressure delivered to the spinal column. Additionally, they maintain the proper anatomical separation between two adjacent vertebrae. This separation is necessary for allowing both the afferent and efferent nerves to exit and enter, respectively, the spinal column. Alternatively, or in addition, there are several types of spinal curvature disorders. Examples of such spinal curvature disorders include, but need not be limited to, lordosis, kyphosis and scoliosis.

One technique of treating spinal disorders, in particular the degenerative, traumatic and/or congenital issues, is via surgical arthrodesis of the spine. This can be accomplished by removing the intervertebral disk and replacing it with implant(s) and/or bone and/or immobilizing the spine to allow the eventual fusion or growth of the bone across the disk space to connect the adjoining vertebral bodies together. The stabilization of the vertebra to allow fusion is often assisted by the surgically implanted device(s) to hold the vertebral bodies in proper alignment and allow the bone to heal, much like placing a cast on a fractured bone. Such techniques have been effectively used to treat the above-described conditions and in most cases are effective at reducing the patient's pain and preventing neurological loss of function.

Complications of joint fusions and/or other procedures of the spine can include those applicable to any surgery such as bone and/or soft-tissue infection, wound dehiscence, and failure of fixation. Other complications which may be more specific to fusion procedures can include malalignment, proximal or distal joint deterioration, and delayed union or nonunion, including potential complications resulting from medical comorbidities, patient noncompliance, and/or inappropriate fixation. Accordingly, there is need for further improvement in surgical implants, and the present subject matter is such improvement.

### BRIEF SUMMARY OF THE INVENTION

The following presents a simplified summary of the subject matter in order to provide a basic understanding of some aspects of the subject matter. This summary is not an extensive overview of the subject matter. It is intended to neither identify key or critical elements of the subject matter nor delineate the scope of the subject matter. Its sole purpose is to present some concepts of the subject matter in a simplified form as a prelude to the more detailed description that is presented later.

In accordance with various aspects of the present subject matter, implant devices and/or components thereof are described that incorporate a variety of surface and/or subsurface features or treatments that can promote bone growth into and/or through the implant, which may be useful in joint and/or bone replacement implants used in spinal surgeries, dental surgeries and/or other orthopedic procedures. In various embodiments, such implants may incorporate silicon nitride (i.e., Si3N4 and/or chemical analogues thereof) in their construction, either in the entirety of the implant as well as components, portions, layers and/or surfaces thereof. In various embodiments, the silicon nitride material(s) will be highly osteo-inductive and/or osteoconductive and will desirably facilitate and/or promote implant fixation to adjacent living bone surfaces, while concurrently reducing and/or inhibiting periprosthetic infection and/or bacterial adhesion to the surfaces and/or interior portions of the implant.

In various applications, the utility of silicon nitride as an implant material can be enhanced by the addition of various other medical materials, including the use of one or various combinations of titanium, chrome cobalt, stainless steel, silicone, poly (ether ether ketone) (PEEK), ultra-high molecular-weight polyethylene (UHMWPE), polyurethane foams, polylactic acid, apatites and/or various 3D printed materials. In such cases, the employment of such material mixtures in implant construction may enhance the strength and/or durability of a desired implant design, as well as allow for improved surgical outcomes and/or greatly reduced complication rates.

If desired, implants can be constructed from a variety of modular components, including modular components comprising different materials. If desired, such modular components could be provided in a kit form for selection and/or assembly in a surgical theatre and/or in situ during a surgical procedure. If desired, various components may be removable and replaceable.

Various surgical methods for preparing anatomical surfaces and/or for implanting or placement of the various devices and/or components described herein are also described, including the insertion and placement of implants between adjacent vertebrae of the spine as well as within bones and/or between other joint surfaces.

In accordance with another aspect of the present subject matter, various methods for manufacturing devices and/or components thereof, as set for within any of the details described with the present application, are provided.

While embodiments and applications of the present subject matter have been shown and described, it would be apparent that other embodiments, applications and aspects are possible and are thus contemplated and are within the scope of this application.

The following description and the annexed drawings set forth in detail certain illustrative aspects of the subject matter. These aspects are indicative, however, of but a few of the various ways in which the principles of the subject matter may be employed and the present subject matter is intended to include all such aspects and their equivalents. Other objects, advantages and novel features of the subject matter will become apparent from the following detailed description of the subject matter when considered in conjunction with the drawings

In accordance with various aspects of the present subject matter, implant components may include bone cement formulations which incorporate silicon nitride (i.e., Si₃N₄ and/or chemical analogues thereof) in their mixtures and/or composition, which may include the incorporation of silicon nitride powders, granules, particulates, portions, pebbles, blocks, layers and/or coatings of solids and/or particulates within the bone cement mixture. In various embodiments, the bone cement may be a liquid, paste, gel or dough, and preferably hardens to a substantially solid solidified material. In at least one exemplary embodiment, a PMMA bone cement formulation comprising a powered MMA-styrene co-polymer and a reactive monomer such as methyl methacrylate can be combined with various percentages by weight and/or volume of a ceramic material such as a silicon nitride material, which when mixed and polymerized can result in a polymerized and/or “cured” block, implant and/or structure capable of implantation in a bony defect and/or other location. In various embodiments, the ceramic material may comprise a granular or regularly/irregularly shaped material, with the granules having a plurality of interconnecting micropores. In various embodiments, a plurality of different sizes of granules may be used.

In at least one embodiment, a PMMA bone cement formulation comprising a powered MMA-styrene co-polymer and a reactive monomer such as methyl methacrylate can be combined with various percentages by weight and/or volume of a powdered, granulated and/or fluidized silicon nitride material, which when mixed can create a flowable and/or moldable material which will desirably harden and/or polymerize into a variety of shapes, which can include injection of the flowable material through a syringe or tube into a void or opening to partially and/or fully fill the void or opening, wherein the material will subsequently harden and/or polymerize into a shape which can be defined by the cavity in which it sits. This could include the injection into various anatomical locations as well as injection and/or introduction into implants and/or other devices prior to, during and/or after their implantation into a targeted patient anatomy (i.e., such as within the graft chamber of an intervertebral fusion implant).

In accordance with various aspects of the present subject matter, bone cements and/or other implants, devices and/or components thereof are described that incorporate silicon nitride (i.e., Si3N4 and/or chemical analogues thereof) in their construction, either in the entirety of the implant as well as components, portions, layers and/or surfaces thereof. In various embodiments, the silicon nitride material(s) will be highly osteo-inductive and/or osteoconductive and will desirably facilitate and/or promote fixation to adjacent living bone surfaces, while concurrently reducing and/or inhibiting periprosthetic infection and/or bacterial adhesion.

In various applications, the utility of silicon nitride as an implant material can be enhanced by the addition of various other medical materials, including the use of one or various combinations of titanium, chrome cobalt, stainless steel, silicone, poly (ether ether ketone) (PEEK), ultra-high molecular-weight polyethylene (UHMWPE), polyurethane foams, polylactic acid, apatites and/or various 3D printed materials. In such cases, the employment of such material mixtures in implant construction may enhance the strength and/or durability of a desired implant design, as well as allow for improved surgical outcomes and/or greatly reduced complication rates.

If desired, implants can be constructed from a variety of modular components, including modular components comprising different materials and/or injectable or formable silicon nitride/PMMA cements. If desired, such modular components could be provided in a kit form for selection and/or assembly in a surgical theatre and/or in situ during a surgical procedure. If desired, various components may be removable and replaceable.

Various surgical methods for preparing anatomical surfaces and/or for implanting or placement of the various devices and/or components described herein are also described, including the insertion and placement of implants between adjacent vertebrae of the spine as well as within bones and/or between bones and/or joint surfaces or other body locations.

In accordance with another aspect of the present subject matter, various methods for manufacturing devices and/or components thereof, as set for within any of the details described with the present application, are provided.

### BRIEF DESCRIPTION OF THE SEVERAL VIEWS OF THE DRAWINGS

The foregoing and other features and advantages of the present subject matter will become apparent to those skilled in the art to which the present subject matter relates upon reading the following description with reference to the accompanying drawings. It is to be appreciated that two copies of the drawings are provided; one copy with notations therein for reference to the text and a second, clean copy that possibly provides better clarity.

FIG. 1 illustrates a portion of a patient's spinal column;

FIG. 2A illustrates a perspective view of an example of a cage structure that is constructed according to the principles of the disclosure;

FIG. 2B illustrates another view of the cage structure illustrated in FIG. 2A;

FIG. 3 depicts an exploded perspective view of one exemplary embodiment of a cage structure comprising a plurality of modular components;

FIGS. 4A and 4B depict a perspective and top plan view of another alternative embodiment of a cage structure comprising a silicon nitride component with a supplemental component comprising a different material;

FIGS. 5A through 5D depict various views of another alternative embodiment of a cage structure comprising a porous silicon nitride body with a metallic insert;

FIG. 5E depicts an alternative embodiment of an anterior plate that can be engaged with an implant body;

FIGS. 5F and 5G depicting alternative embodiments of implant constructs with associated fixation screws;

FIGS. 6A through 6D depict various views of another alternative embodiment of a cage structure comprising a porous machined silicon nitride body;

FIG. 7A depicts a perspective view of another alternative embodiment of a cage structure with a silicon nitride implant body and an anterior engagement plate;

FIG. 7B depicts a cross-sectional view of a sacrum and an ilium, with the cage structure of FIG. 7A implanted therebetween;

FIG. 8A depicts a perspective view of another alternative embodiment of a cage structure with a silicon nitride implant body and a plurality of support wires or walls;

FIG. 8B depicts an exemplary wireframe-type structure;

FIG. 8C depicts a supporting wall or filament support structure provided proximate to a fusion visualization window;

FIG. 9A depicts another exemplary embodiment of a cage structure with a silicon nitride or PEEK implant body and a silicon nitride plug;

FIG. 9B depicts an alternative embodiment of a cage structure including a silicon nitride or PEEK or titanium implant body with a hollow cylinder positioned in a central cavity;

FIG. 9C depicts another alternative embodiment of a cage structure including at least one recess;

FIG. 10 depicts various cross-sectional views of a spinal implant with various exemplary silicon nitride insert geometries formed therein;

FIG. 11 depicts exemplary degrees of hydrophobicity for various medical grade materials, including silicon nitride;

FIGS. 12A and 12 B depict cross-sectional views of silicon nitride implants with neovascularization induced within the porous sections of the implant;

FIGS. 13A through 13C depict three exemplary implants made of PEEK, Titanium and silicon nitride and their effects on adjacent living bone;

FIG. 13D depicts a magnetic field induced by a bar-type magnet;

FIG. 13E depicts the effect of silicon nitride material on new bone growth;

FIGS. 14A through 14C depict exemplary effects of a silicon nitride surface on bacteria near the implant;

FIGS. 15A and 15B depict views of another exemplary embodiment of a surgical implant that incorporates silicon nitride features to enhance osseous integration and/or improve bacterial resistance;

FIG. 16A through 16E depict another exemplary embodiment of a surgical implant that incorporates silicon nitride features to enhance osseous integration and/or improve bacterial resistance; and

FIGS. 17A though 17G depict various views of a rotation resistant spinal rod and screw system.

FIG. 18 illustrates a cross-sectional view of a vertebral bone filled with a PMMA cement;

FIG. 19 illustrates a cross-sectional view of a vertebral bone filled with one exemplary embodiment of a silicon nitride cement;

FIGS. 20A and 20B depict perspective views of cement structures incorporating resorbable silicon nitride granules;

FIGS. 21A and 21B depict the cement structures of FIGS. 20A and 20B after absorption; of some silicon nitride;

FIG. 22A depicts a perspective view of a silicon nitride agglomeration in a cement formulation;

FIG. 22B depicts various exemplary geometries for resorbable silicon nitride granules mixed into a PMMA cement formulation to enhance macro porosity;

FIGS. 23A through 23C depict various exemplary silicon nitride granular shapes;

FIG. 24 depicts an exemplary grain size distribution for silicon nitride granules for use in cement formulations;

FIGS. 25A and 25B depict SEM photographs of an exemplary PMMA cement incorporating resorbable ceramic granules;

FIG. 25C depicts an exemplary ceramic granule with associated PMMA cement;

FIGS. 26A through 26F depict various desirable attributes of an implant comprising a silicon nitride cement.

FIG. 27A depicts another exemplary embodiment of a cage structure incorporating a plurality of different surface features which enhance bone growth;

FIG. 27B depicts a visually detectable rough surface feature of the cage structure of FIG. 27A;

FIG. 27C depicts a magnified view of the visually detectable rough surface feature of FIG. 27B;

FIG. 27D depicts a visually detectable smooth surface feature of the cage structure of FIG. 27A;

FIG. 28A depicts an external surface of an exemplary implant wherein white indicates peaks while black indicates valleys in the surface texture;

FIG. 28B depicts a magnified view of various external surface features of FIG. 27B;

FIG. 29A depicts various body cells involved in the process of ossification;

FIG. 29B depict a highly magnified view of osteocyte bone cells;

FIG. 30 depicts a variety of exemplary treatments and/or feature densities that can be incorporated into an implant design;

FIG. 31 depicts various existing manufacturing techniques that can be utilized to create surface features described herein;

FIG. 32 depicts an exemplary capillary-like effect on body cells induced by the proposed surface features described herein;

FIGS. 33A and 33B depict perspective and top plan views of one exemplary embodiment of a cage structure with an insertable/removeable plug;

FIGS. 34A and 34B depict perspective views of another exemplary embodiment of a cage structure with an insertable/removeable plug;

FIGS. 35A and 35B depict a variety of geometric and dimensional features than can be incorporated into plugs; and

FIG. 35C depicts a top plan view of various commercially available implants with different shape and/or size openings therein.

The following description and the annexed drawings set forth in detail certain illustrative aspects of the subject matter. These aspects are indicative, however, of but a few of the various ways in which the principles of the subject matter may be employed and the present subject matter is intended to include all such aspects and their equivalents. Other objects, advantages and novel features of the subject matter will become apparent from the following detailed description of the subject matter when considered in conjunction with the drawings.

### DETAILED DESCRIPTION OF THE INVENTION

The disclosure and the various features and advantageous details thereof are explained more fully with reference to the non-limiting embodiments and examples that are described and/or illustrated in the accompanying drawings and detailed in the following description. It should be noted that the features illustrated in the drawings are not necessarily drawn to scale, and features of one embodiment may be employed with other embodiments as the skilled artisan would recognize, even if not explicitly stated herein. Descriptions of well-known components and processing techniques may be omitted so as to not unnecessarily obscure the embodiments of the disclosure. The examples used herein are intended merely to facilitate an understanding of ways in which the disclosure may be practiced and to further enable those of skill in the art to practice the embodiments of the disclosure. Accordingly, the examples and embodiments herein should not be construed as limiting the scope of the disclosure. Moreover, it is noted that like reference numerals represent similar parts throughout the several views of the drawings.

The terms “including,” “comprising” and variations thereof, as used in this disclosure, mean “including, but not limited to,” unless expressly specified otherwise. The terms “a,” “an,” and “the,” as used in this disclosure, mean “one or more,” unless expressly specified otherwise.

Devices and/or device components that are disclosed in communication with each other need not necessarily be in continuous communication with each other, unless expressly specified otherwise. In addition, devices that are in direct contact with each other may contact each other directly or indirectly through one or more intermediary articles or devices. The device(s) disclosed herein may be made of a material such as silicon nitride, which may alternatively be combined, in various embodiments, with other materials such as, for example, a polymer, a metal, an alloy, or the like. For instance, the device(s) may comprise silicon nitride, alone or in combination with a Polyether Ether Ketone (PEEK), titanium, a titanium alloy, or the like, or various combinations of the foregoing. The material may be formed by a process such as, for example, an active reductive process of a metal (e.g., titanium or titanium alloy) to increase the amount of nanoscaled texture to device surface(s), so as to increase promotion of bone growth and fusion.

Although process steps, method steps, or the like, may be described in a sequential order, such processes and methods may be configured in alternate orders. In other words, any sequence or order of steps that may be described does not necessarily indicate a requirement that the steps be performed in that order. The steps of the processes or methods described herein may be performed in any order practical. Further, some steps may be performed simultaneously.

When a single device or article is described herein, it will be readily apparent that more than one device or article may be used in place of a single device or article. Similarly, where more than one device or article is described herein, it will be readily apparent that a single device or article may be used in place of the more than one device or article. The functionality or the features of a device or article may be alternatively embodied by one or more other devices or articles which are not explicitly described as having such functionality or features.

Certain exemplary embodiments will now be described to provide an overall understanding of the principles of the structure, function, manufacture, and use of the devices and methods disclosed herein. One or more examples of these embodiments are illustrated in the accompanying drawings. Those of ordinary skill in the art will understand that the devices and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

The present invention provides various devices, systems and methods for treating various anatomical structures of the spine and/or other areas of human and/or animal bodies. While the disclosed embodiments may be particularly well suited for use during surgical procedures for the repair, fixation and/or support of vertebrae, it should be understood that various other anatomical locations of the body may benefit from various features of the present invention.

The spinal column of vertebrates provides support to bear weight and protection to the delicate spinal cord and spinal nerves. The spinal column includes a series of vertebrae stacked on top of each other. There are typically seven cervical (neck), twelve thoracic (chest), and five lumbar (low back) segments. Each vertebra has a cylindrical shaped vertebral body in the anterior portion of the spine with an arch of bone to the posterior, which covers the neural structures. Between each vertebral body is an intervertebral disk, a cartilaginous cushion to help absorb impact and dampen compressive forces on the spine. To the posterior, the laminar arch covers the neural structures of the spinal cord and nerves for protection. At the junction of the arch and anterior vertebral body are articulations to allow movement of the spine.

Various types of problems can affect the structure and function of the spinal column. These can be based on degenerative conditions of the intervertebral disk or the articulating joints, traumatic disruption of the disk, bone or ligaments supporting the spine, tumor or infection. In addition, congenital or acquired deformities can cause abnormal angulation or slippage of the spine. Anterior slippage (spondylolisthesis) of one vertebral body on another can cause compression of the spinal cord or nerves. Patients who suffer from one of more of these conditions often experience extreme and debilitating pain, and can sustain permanent neurological damage if the conditions are not treated appropriately. Alternatively or in addition, there are several types of spinal curvature disorders. Examples of such spinal curvature disorders include, but need not be limited to, lordosis, kyphosis and scoliosis.

One technique of treating spinal disorders, in particular the degenerative, traumatic and/or congenital issues, is via surgical arthrodesis of the spine. This can be accomplished by removing the intervertebral disk and replacing it with implant(s) and/or bone and immobilizing the spine to allow the eventual fusion or growth of the bone across the disk space to connect the adjoining vertebral bodies together. The stabilization of the vertebra to allow fusion is often assisted by the surgically implanted device(s) to hold the vertebral bodies in proper alignment and allow the bone to heal, much like placing a cast on a fractured bone. However, while such techniques have been effectively used to treat the above-described conditions and can be effective at reducing the patient's pain and preventing neurological loss of function, current bone implants (1) typically play a passive role as a bone replacement structure and do not contribute significantly to bone growth, (2) often create interference, distortion and/or imaging artifacts during non-invasive imaging of a treated anatomical region, (3) often require a blood pathway between bridging elements, such as by requiring the end user to ensure that proper amounts of blood conducting agents have been added and/or packed along and/or around the implant, (4) often have porous openings, tessellated supports and/or other structural features that are difficult to fabricate and that require specialized or expensive fabrication methods, (5) are often made of materials such as metals or polymers that do not induce bone growth or only allow a thin film of bone to adhere to the implant materials, and (6) do not provide anti-bacterial properties. Because of this, such procedures are still associated with significant levels of surgical complications such as non-unions and/or infections, and thus there is need for further improvement. The present subject matter is such improvement.

The present subject matter will now be described with reference to the drawings, wherein like reference numerals are used to refer to like elements throughout. It is to be appreciated that the various drawings are not necessarily drawn to scale from one figure to another nor inside a given figure, and in particular that the size of the components may be arbitrarily drawn for facilitating the understanding of the drawings. In the following description, for purposes of explanation, numerous specific details are set forth in order to provide a thorough understanding of the present subject matter. It may be evident, however, that the present subject matter can be practiced without these specific details. Additionally, other embodiments of the subject matter are possible and the subject matter is capable of being practiced and carried out in ways other than as described. The terminology and phraseology used in describing the subject matter is employed for the purpose of promoting an understanding of the subject matter and should not be taken as limiting.

In various embodiments, the implants and/or portions may comprise silicon nitride and/or various combinations of a variety of surgically acceptable materials, including radiopaque and/or radiolucent materials, other materials or combinations of such materials. Radiolucent materials can include, but are not limited to, polymers, carbon composites, fiber-reinforced polymers, plastics, combinations thereof and the like. Radiopaque materials are traditionally used to construct devices for use in the medical device industry. Radiopaque materials can include, but are not limited to, metal, aluminum, stainless steel, titanium, titanium alloys, cobalt chrome alloys, combinations thereof and the like.

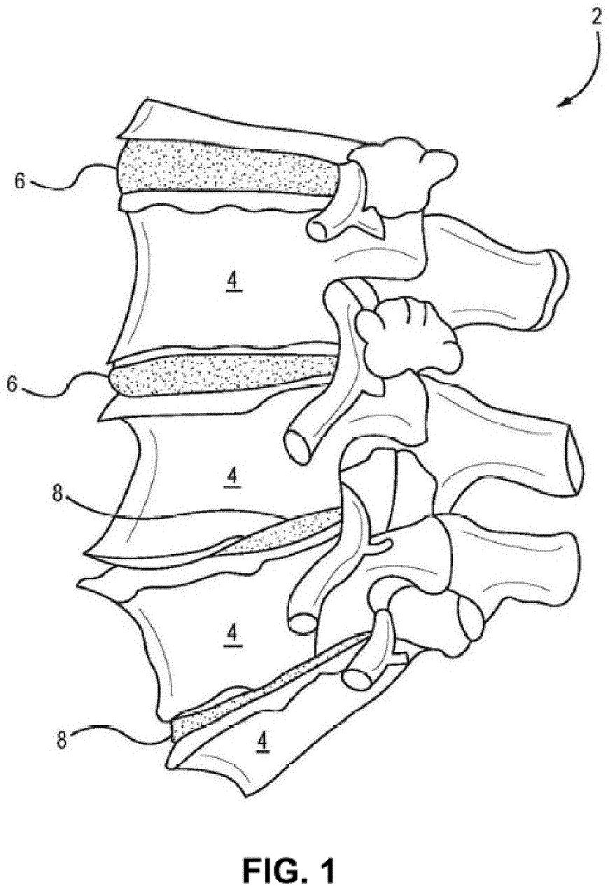

FIG. 1 depicts a portion of a patient's spinal column 2, including vertebrae 4 and intervertebral discs 6. In humans, the spinal column is generally formed by individual interlocking vertebrae, which are classified into five segments, including (from head to tail) a cervical segment (vertebrae C1-C7), a thoracic segment (vertebrae T1-T12), a lumbar segment (vertebrae L1-L5), a sacrum segment (vertebrae S1-S5), and coccyx segment (vertebrate Co1-Co5). The cervical segment forms the neck, supports the head and neck, and allows for nodding, shaking and other movements of the head. The thoracic segment attaches to ribs to form the ribcage. The lumbar segment carries most of the weight of the upper body and provides a stable center of gravity during movement. The sacrum and coccyx make up the back walls of the pelvis.

Intervertebral discs are located between each of the movable vertebra. Each intervertebral disc typically includes a thick outer layer called the disc annulus, which includes a crisscrossing fibrous structure, and a disc nucleus, which is a soft gel-like structure located at the center of the disc. The intervertebral discs function to absorb force and allow for pivotal movement of adjacent vertebra with respect to each other.

In the vertebral column, the vertebrae increase in size as they progress from the cervical segment to the sacrum segment, becoming smaller in the coccyx. At maturity, the five sacral vertebrae typically fuse into one large bone, the sacrum, with no intervertebral discs. The last three to five coccygeal vertebrae (typically four) form the coccyx (or tailbone). Like the sacrum, the coccyx does not have any intervertebral discs.

Each vertebra is an irregular bone that varies in size according to its placement in the spinal column, spinal loading, posture and pathology. While the basic configuration of vertebrae varies, every vertebra has a body that consists of a large anterior middle portion called the centrum and a posterior vertebral arch called the neural arch. The upper and lower surfaces of the vertebra body give attachment to intervertebral discs. The posterior part of a vertebra forms a vertebral arch that typically consists of two pedicles, two laminae, and seven processes. The laminae give attachment to the ligament flava, and the pedicles have a shape that forms vertebral notches to form the intervertebral foramina when the vertebrae articulate. The foramina are the entry and exit passageways for spinal nerves. The body of the vertebra and the vertical arch form the vertebral foramen, which is a large, central opening that accommodates the spinal canal that encloses and protects the spinal cord.

The body of each vertebra is composed of cancellous bone that is covered by a thin coating of cortical bone. The cancellous bone is a spongy type of osseous tissue, and the cortical bone is a hard and dense type of osseous tissue. The vertebral arch and processes have thicker coverings of cortical bone.

The upper and lower surfaces of the vertebra body are flattened and rough. These surfaces are the vertebral endplates that are in direct contact with the intervertebral discs. The endplates are formed from a thickened layer of cancellous bone, with the top layer being denser. The endplates contain adjacent discs and evenly spread applied loads. The endplates also provide anchorage for the collagen fibers of the disc. Each disc 6 comprises a fibrous exterior surrounding an inner gel-like center which cooperate to distribute pressure evenly across each disc 6, thereby preventing the development of stress concentrations that might otherwise damage and/or impair vertebrae 4 of spinal column 2. Discs 6 are, however, subject to various injuries and/or disorders which may interfere with a disc's ability to adequately distribute pressure and protect vertebrae 4. For example, disc herniation, degeneration, and infection of discs 6 may result in insufficient disc thickness and/or support to absorb and/or distribute forces imparted to spinal column 2. Disc degeneration, for example, may result when the inner gel-like center begins to dehydrate, which may result in a degenerated disc 8 having decreased thickness. This decreased thickness may limit the ability of degenerated disc 8 to absorb shock which, if left untreated, may result in pain and/or vertebral injury

While pain medication, physical therapy, and other non-operative conditions may alleviate some symptoms, such interventions may not be sufficient for every patient. Accordingly, various procedures have been developed to surgically improve patient quality of life via abatement of pain and/or discomfort. Such procedures may include, discectomy and fusion procedures, such as, for example, anterior cervical interbody fusion (ACIF), anterior lumbar interbody fusion (ALIF), direct lateral interbody fusion (DLIF) (also known as XLIF), posterior lumbar interbody fusion (PLIF), and transforaminal lumbar interbody fusion (TLIF). During a discectomy, all or a portion of a damaged disc (for example, degenerated disc 8, shown in FIG. 1), is removed via an incision, typically under X-ray guidance.

As previously noted, the various implant devices and/or components thereof disclosed herein can optionally incorporate a silicon nitride material (i.e., Si₃N₄ and/or chemical analogues thereof) in their construction, either in the entirety of the implant as well as components, portions, layers and/or surfaces thereof. The incorporation of silicon nitride as a component material for spinal or other implants can provide significant improvements over existing implant materials and material designs currently available, as the silicon nitride material(s) will be highly osteo-inductive and/or osteoconductive and will desirably facilitate and/or promote implant fixation to adjacent living bone surfaces, while concurrently reducing and/or inhibiting periprosthetic infection and/or bacterial adhesion to the surfaces and/or interior portions of the implant

Silicon nitride (Si₃N₄) and its various analogs can impart both antibacterial and osteogenic properties to an implant, including to bulk Si₃N₄ as well as to implants coated with layers of Si₃N₄ of varying thicknesses. In bone replacement as well as prosthetic joint fusion and/or replacement, osseous fixation of implants through direct bone ingrowth (i.e., cementless fixation) is often preferred, and such is often attempted using various surface treatments and/or the incorporation of porous surface layers (i.e., porous Ti6Al4V alloy) on one or more bone-facing surfaces of an implant. Silicon nitride surfaces express reactive nitrogen species (RNS) that promote cell differentiation and osteogenesis, while resisting both gram-positive and gram-negative bacteria. This dual advantage of RNS in terms of promoting osteogenesis, while discouraging bacterial proliferation, can be of significant utility in a variety of implant designs.

Desirably, the inclusion of silicon nitride components into a given implant design will encompass the use of bulk silicon nitride implants, as well as implants incorporating other materials that may also include silicon nitride components and/or layers therein, with the silicon nitride becoming an active agent of bone fusion. RNS such as N2O, NO, and —OONO are highly effective biocidal agents, and the unique surface chemistries of Si₃N₄ facilitate its activity as an exogenous NO donor. Spontaneous RNS elution from Si₃N₄ discourages surface bacterial adhesion and activity, and unlike other direct eluting sources of exogenous NO, Si₃N₄ elutes mainly NH₄⁺ and a small fraction of NH3 ions at physiological pH, because of surface hydrolysis and homolytic cleavage of the Si—N covalent bond. Ammonium NH₄⁺ can enter the cytoplasmic space of cells in controlled concentrations and through specific transporters, and is a nutrient used by cells to synthesize building-block proteins for enzymes and genetic compounds, thus sustaining cell differentiation and proliferation. Together with the leaching of orthosilicic acid and related compounds, NH₄⁺ promotes osteoblast synthesis of bone tissue and stimulates collagen type 1 synthesis in human osteoblasts. Conversely, highly volatile ammonia NH₃ can freely penetrate the external membrane and directly target the stability of DNA/RNA structures in bacterial cells. However, the release of unpaired electrons from the mitochondria in eukaryotic cells activates a cascade of consecutive reactions, which starts with NH3 oxidation into hydroxylamine NH₂OH (ammonia monooxygenase) along with an additional reductant contribution leading to further oxidation into NO₂— nitrite through a process of hydroxylamine oxidoreductase. This latter process involves nitric oxide NO formation. In Si₃N₄, the elution kinetics of such nitrogen species is slow but continuous, thus providing long-term efficacy against bacterial colonies including mutants (which, unlike eukaryotic cells, lack mitochondria). However, when slowly delivered, NO radicals have been shown to act in an efficient signaling pathway leading to enhanced differentiation and osteogenic activity of human osteoblasts. Desirably, Si₃N₄ materials can confer resistance against adhesion of both Gram-positive and Gram-negative bacteria, while stimulating osteoblasts to deposit more bone tissue, and of higher quality.

Where the presence of bulk silicon nitride implant materials may not be desired and/or may be impractical for some reason, it may be desirous to incorporate modules and/or layers including silicon nitride on other materials. Silicon nitride structures and/or components can be formed using a variety of techniques, including by compressing, milling and firing silicon nitride powder, as well as by extruding silicon nitride into sheet, tube, pipe and/or thread form (which may be further processed into thread or “rope” by braiding and/or other techniques). Silicon nitride shapes may also be manufactured using subtractive manufacturing techniques (i.e., machining, milling and/or surface roughening), as well as by using additive manufacturing techniques (i.e., surface coating, brazing, welding, bonding, deposition on various material surfaces and/or even by 3D laser printing of structures). If desired, silicon nitride may even be formed using curing or other light/energy activation techniques, such as where a slurry of liquid polymer and silicon nitride particles may be UV cured to create a 3-dimensional structure and/or layer containing silicon nitride. In various embodiments, silicone nitride may be utilized in block form, in sheets, columns and bars, in cable or braided form, in mesh form, in a textured surface coating, in powder form, in granular form, in gel, in putty, in foams and/or as a surface filler and/or coating. In some cases, a surface layer of silicon nitride may be formed on an external and/or internal surface of an implant.

For example, in some embodiments it may be desirous to laser-sinter a thin layer of silicon nitride material (i.e., powder) to the surface of another material, such as PEEK or titanium. One exemplary starting micrometric powder used for laser-sintering of a Si₃N₄ coating in this manner could comprise a 90 wt % fraction of Si₃N₄ powder mixed with a 6 wt % of yttrium oxide (Y₂O₃) and a 4 wt % of aluminum oxide (Al₂O₃). If desired, a Vision LWI VERGO-Workstation equipped with a Nd:YAG laser with a wavelength of 1064 nm (max pulse energy: 70 J, peak power 17 kW, voltage range 160-500 V, pulse time 1-20 ms, spot size 250-2000 μm) can be utilized to achieve densification of successive layers of Si₃N₄ powder placed on a water-wet surface of a Titanium substrate in a nitrogen environment, which desirably limits Si₃N₄ decomposition and oxidation. In the exemplary embodiment, the Nd:YAG laser can be pulsed with a spot size of 2 mm, and driven by an applied voltage of 400 V with a pulse time of 4 ms. This operation can be repeated until a continuous thickness of 15 μm (±5 μm) is formed over an entire surface of the Titanium substrate. This process can create a wavy morphology of the ceramic/metal interface, with interlocks at the micrometer scale between metal and ceramic phases and desirably little or no diffusional transport of the Titanium element into the coating during laser sintering.

In various embodiments, the properties of the disclosed implants will desirably include improvements in one or more of the following: (1) Flexibility in manufacturing and structural diversity, (2) Strong, tough and reliable constructs, (3) Phase stable materials, (4) Favorable imaging characteristics, (5) Hydrophilic surfaces and/or structures, (6) Osteoconductive, (7) Osteoinductive, and/or (8) Anti-Bacterial characteristics.

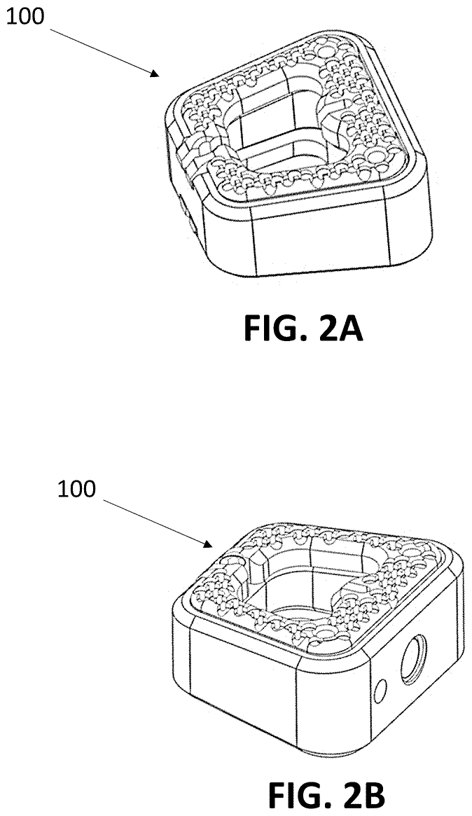

FIGS. 2A and 2B illustrate various views of one exemplary embodiment of a cage structure 100 that can be constructed according to various principles of the disclosure, with FIG. 2A illustrating a perspective view of the cage structure 100, and FIG. 2B illustrating another view of the cage structure 100. The cage structure 100 may be constructed as one, two, three, or more parts. The cage structure 100 may comprise a silicon nitride material, which may be combined in various embodiments with other materials such as, for example, a polymer, a metal, an alloy, or the like. For instance, the cage structure 100 may comprise a central block structure made of PEEK, UHWMPE, titanium, chrome cobalt, stainless steel, a titanium alloy, or the like, with one or more outer surface layer(s) of silicon nitride to desirably increase promotion of bone growth and/or fusion in bone-contacting portions of the implant, as well as desirably reducing the potential for bacterial infection of the implant and/or the surgical site.

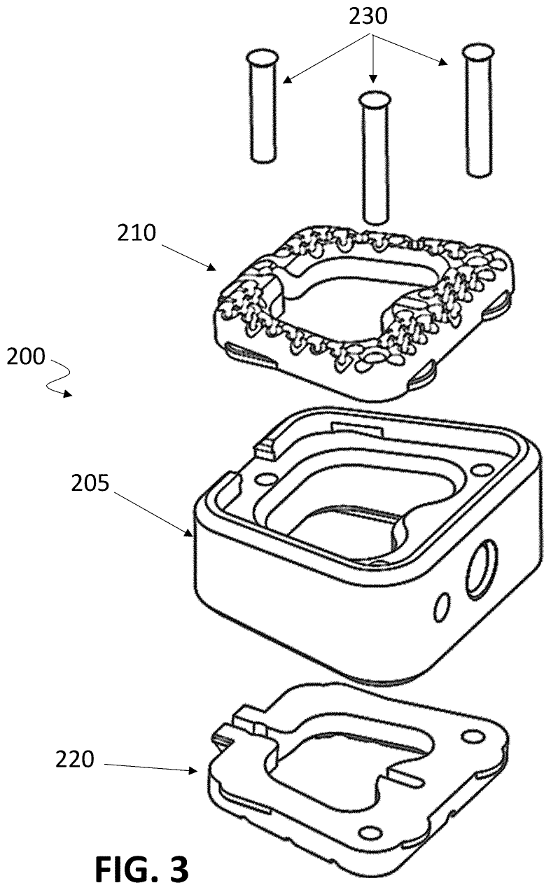

FIG. 3 depicts an exploded perspective view of one exemplary embodiment of a cage structure 200 comprising a plurality of modular components. In this embodiment, a central body 205 has a modular upper surface plate 210 and a modular lower surface plate 220, wherein the various components can be secured together using pins 230 or similar securement components. If desired, the central body could comprise a PEEK material or similar material, with one or both of the modular plates 210 and 220 comprising a silicon nitride material. Alternatively, one or more of the plates 210 and 220 could comprise a titanium material having at least one externally facing surface layer of silicon nitride.

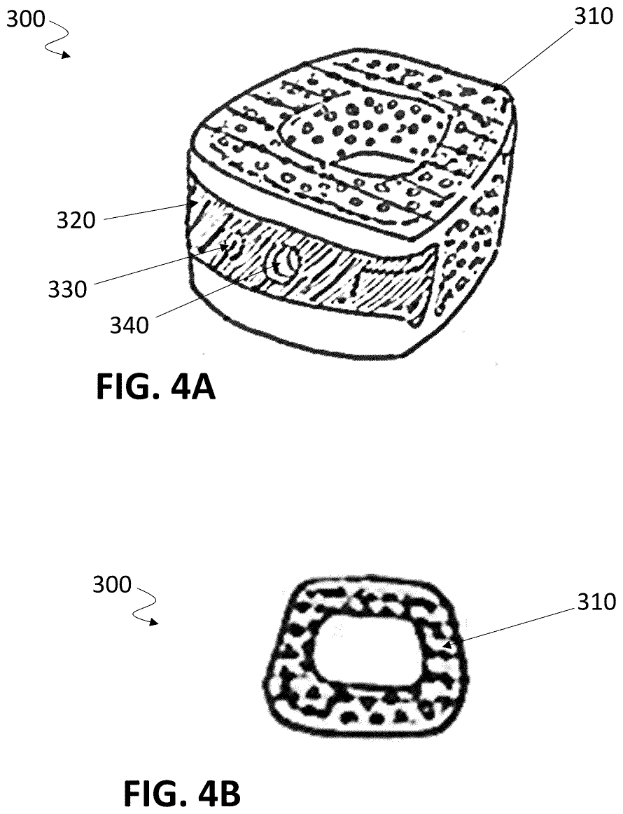

FIGS. 4A and 4B depict a perspective and top plan view of another alternative embodiment of a cage structure 300 comprising a silicon nitride component with a supplemental component comprising a different material. In this embodiment, the main body 310 of the cage structure 300 comprises a silicon nitride block, with an insert 320 comprising a metallic material such as titanium. The insert can include one or more instrument holes 330 to accommodate a surgical insertion and/or placement tool (not shown) and may also include a fixation opening 340 to accommodate a fixation screw (not shown) or removal instrument. Desirably, the insert 320 can function as a gripping point for the cage structure 300, and can also distribute impact or other loading on the silicone nitride block during insertion, adjustment and/or removal of the block in the intervertebral space.

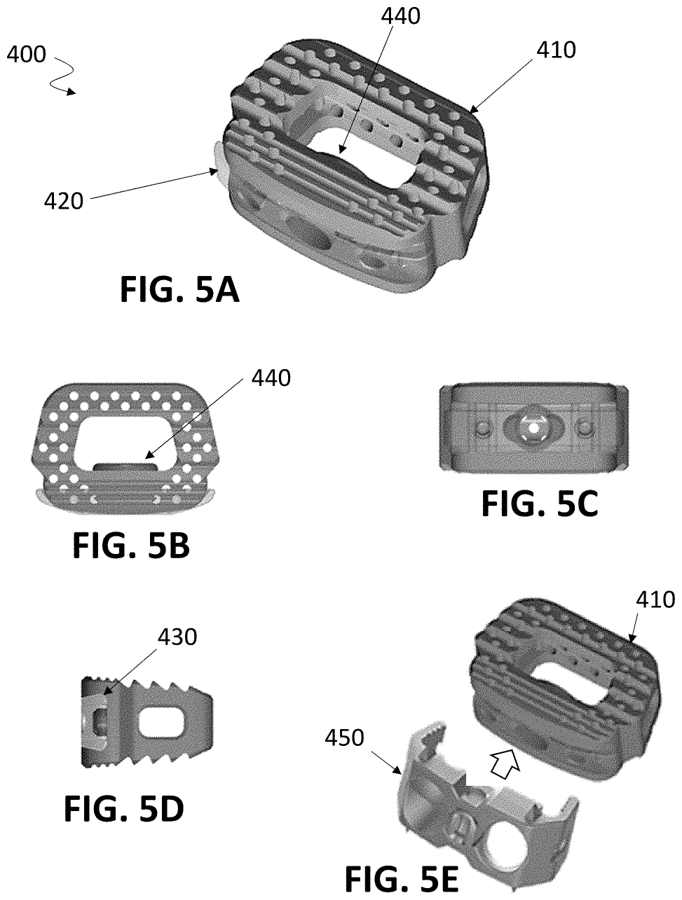

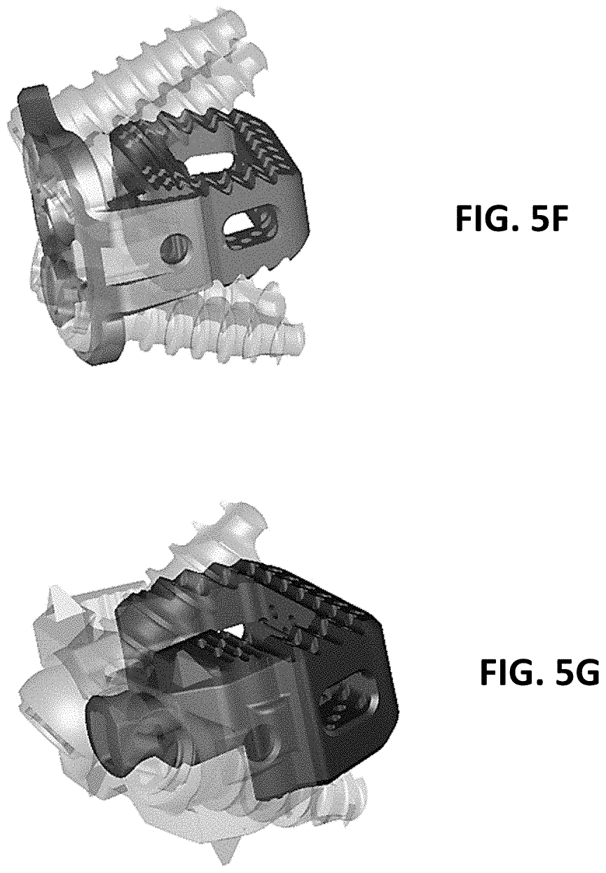

FIGS. 5A through 5D depict various views of another alternative embodiment of a cage structure 400 comprising a porous silicon nitride body 410 with a metallic insert 420. As best seen in FIG. 5D, the insert can desirably slide laterally into a dovetail slot 430 formed in an anterior portion of the body 410, and then the insert can be secured in a desired position using a fixation screw 440. In some embodiments, the fixation screw 440 can comprise an internally threaded shim, which can be utilized with a variety of surgical tools for insertion, adjustment and/or removal of the cage structure 400. FIG. 5E depicts an alternative embodiment of an anterior plate 450 that can be engaged with the body 410, if desired, with FIGS. 5F and 5G depicting various implant constructs and associated fixation screws.

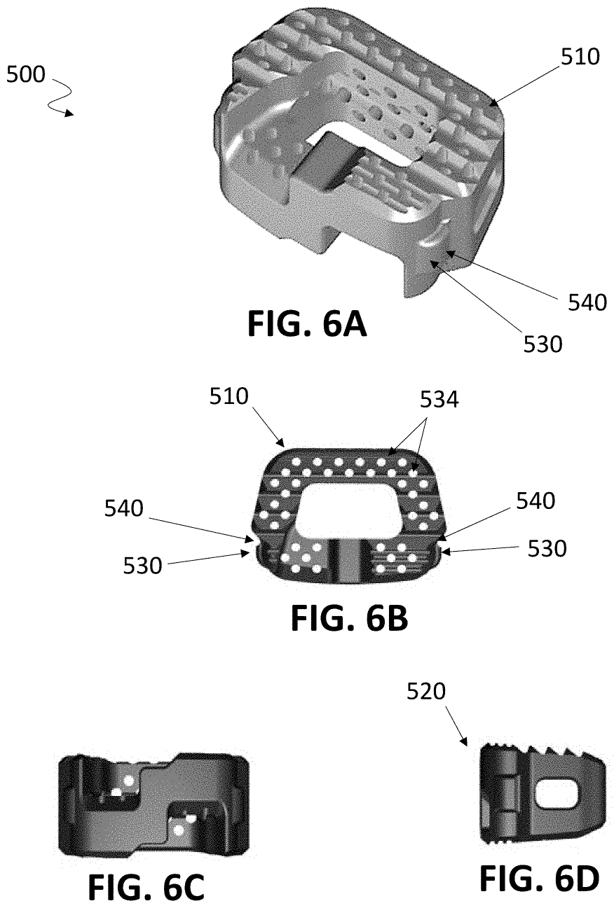

FIGS. 6A through 6D depict various views of another alternative embodiment of a cage structure 500, comprising a porous machined silicon nitride body 510. In this embodiment, an anterior portion 520 of the body 510 can include one or more engagement surfaces 530 and/or engagement depressions 540, which can desirably engage with and/or accommodate one or more corresponding securement feature of an anterior plate 450 (see FIG. 5E) or similar component, which can be attached to the body 510 in a desired manner. Also depicted are a series of openings such as through and/or partial depth holes 534 which can be formed in a variety of ways, including by drilling and/or machining.

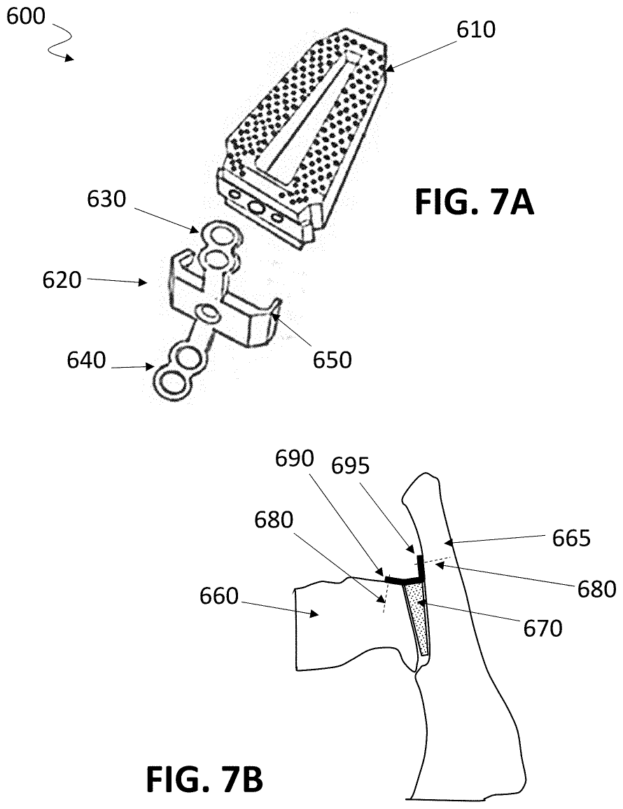

FIG. 7A depicts a perspective view of another alternative embodiment of a cage structure 600, with a silicon nitride implant body 610 and an anterior engagement plate 620. This embodiment, which may be particularly well suited for use in sacral-iliac surgical procedures, can include one or more malleable and/or flexible elements 630, 640, and in some embodiments may optionally include a malleable plate body 650 or portions thereof. FIG. 7B depicts a cross-sectional view of a sacrum 660 and an ilium 665, with one exemplary cage structure 670 implanted therebetween. In this embodiment, the cage is anchored with fixation screws 680 into the underlying bone, with malleable elements 690 and 695 adjusted to match the underlying anatomical surfaces of the adjacent sacrum and ilium into which they are anchored.

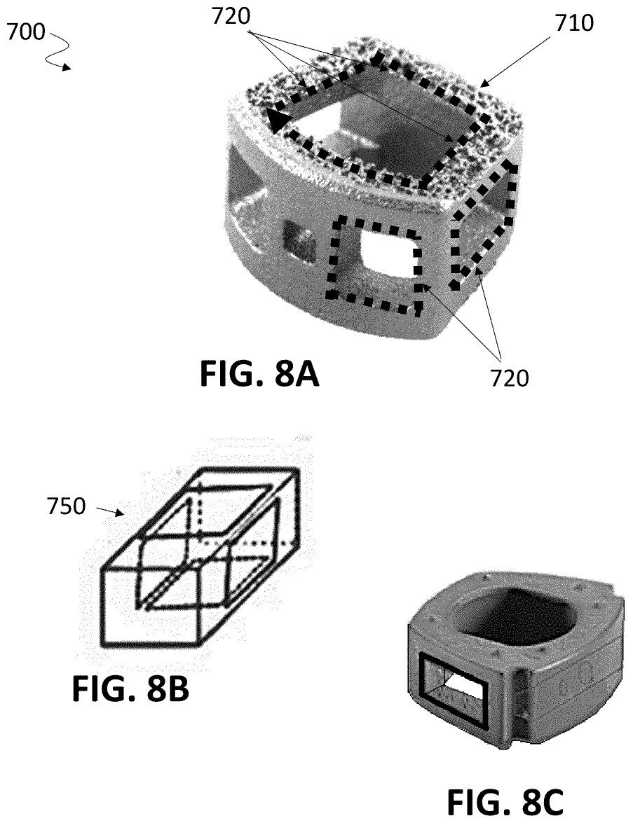

FIG. 8A depicts a perspective view of another alternative embodiment of a cage structure 700, with a silicon nitride implant body 710 and a plurality of support wires or walls 720 which can provide supplemental support to the implant body 710. In his embodiment, the walls 720 (indicated as dotted lines in FIG. 8A) can comprise individual filaments, wires, tubes and/or planar support structures added to the body 710 after formation of the body, or the walls can optionally comprise a wireframe-type structure 750 (i.e., a 2 or 3 dimensional wireframe structure) which may be formed prior to, during and/or after the body is formed. In such a case the wireframe could comprise a framework (see FIG. 8B) upon which a silicon nitride precursor may be positioned and/or deposited, with final firing and/or other treatment of the material may be performed to create a final implant. In various embodiments, walls or filaments or similar support structures can be provided proximate to and/or around various openings in the implant body, including around fusion visualization windows (i.e., at the medial and/or lateral sides of the body—See FIG. 8C) or around some portion or all of the central graft opening.

In other exemplary embodiments, silicon nitride materials of differing compositions and/or states (i.e., solid, liquid and/or flowable or moldable “slurry” states, for example) could be utilized in a single implant and/or portions thereof, including the use of solid silicon nitride for an arthroplasty cage implant, with a moldable silicon nitride “paste” placed within a centrally positioned “graft chamber” of the implant.

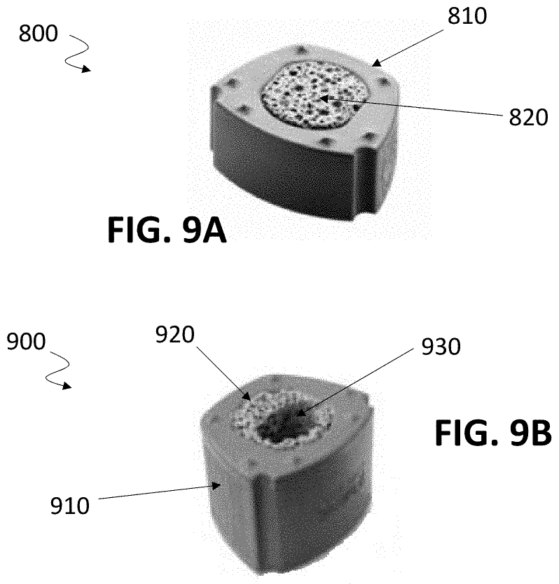

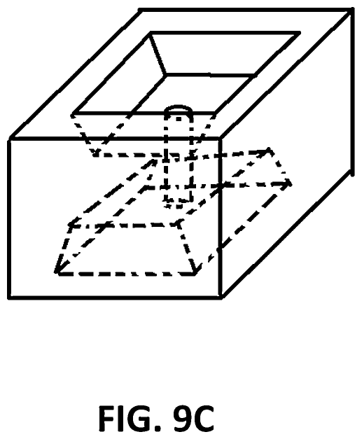

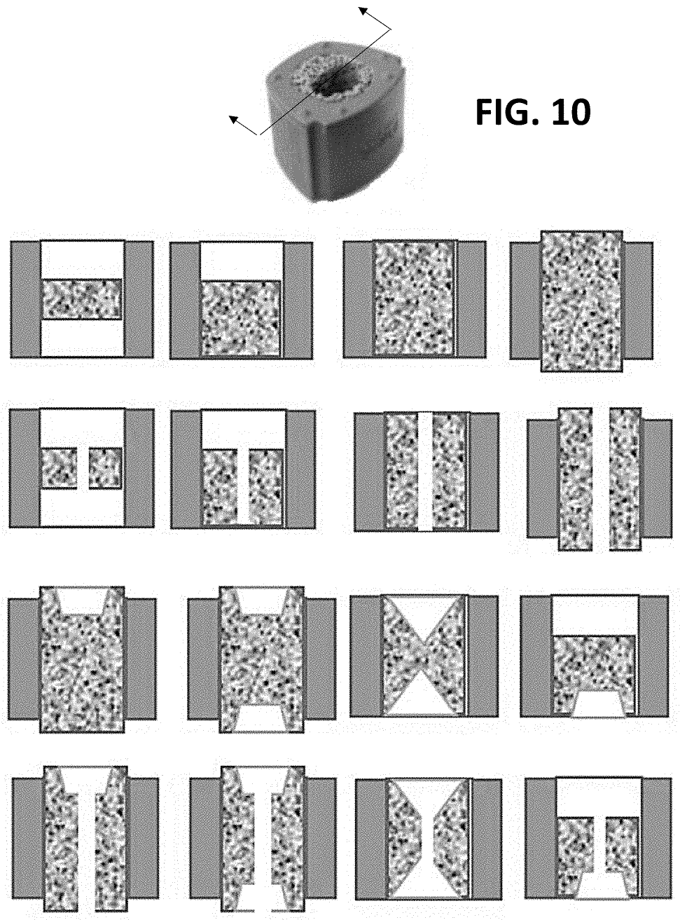

FIG. 9A depicts another exemplary embodiment of a cage structure 800, with a PEEK implant body 810 (or alternatively a silicon nitride body or portions thereof), wherein a silicon nitride plug 820 is positioned in a central cavity of the body 810. If desired, the plug 820 may be formed from a monolithic solid block of silicon nitride, or the plug could comprise a putty or gel containing silicon nitride. In another embodiment shown in FIG. 9B, the cage structure 900 includes a PEEK or titanium implant body 910 (and/or a silicon nitride body or portions thereof), with a hollow cylinder 920 positioned in a central cavity of the body 910. In this embodiment, the opening 930 in the cylinder may be further filled with bone graft or other materials, if desired. In various design alternatives, a cage structure could comprise a central block with a recess in at least one planar direction to facilitate visual of a bone graft packing area. Further alternatives could include at least one recess with a through hole (See FIG. 9C). Various geometry blocks could be made to the pre-specified inner bore geometry and height of a given commercially available implant design, with these blocks fitting into the existing implant to confer the various benefits of silicon nitrite activity thereto. The dimensions of a given silicon nitride block could be smaller than one or more dimensions of the existing implant, or the block could be tapered or straight to facilitate insertion into the implant. If desired, a silicon nitride block or similar component could extend completely through an implant, or only extend partially into and/or out of an implant. FIG. 10 depicts various cross-sectional views of a spinal implant with various exemplary silicon nitride insert geometries formed therein.

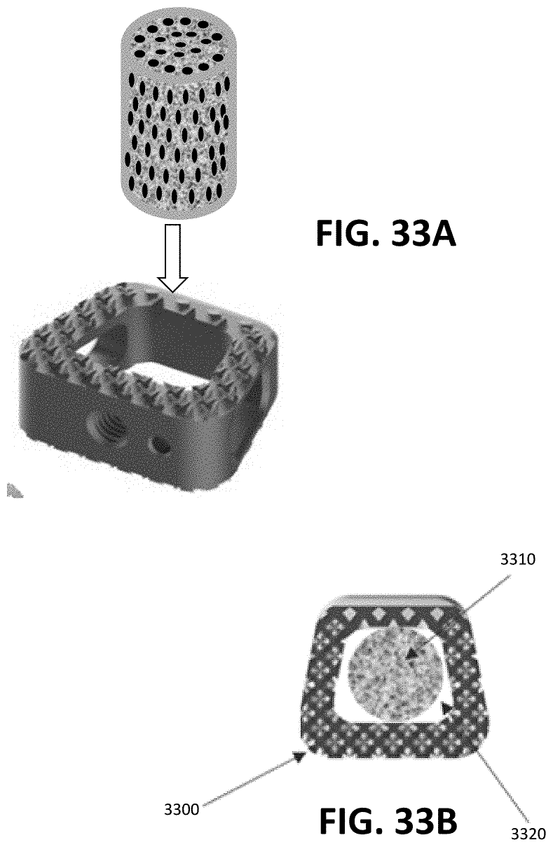

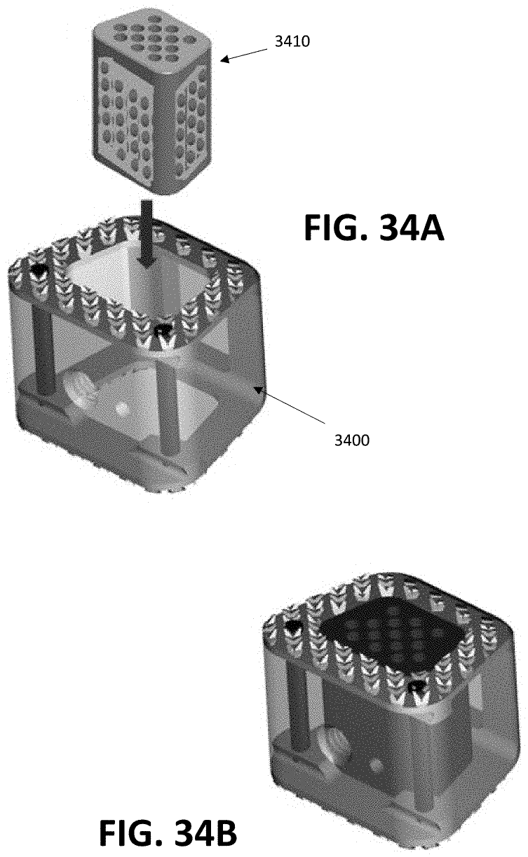

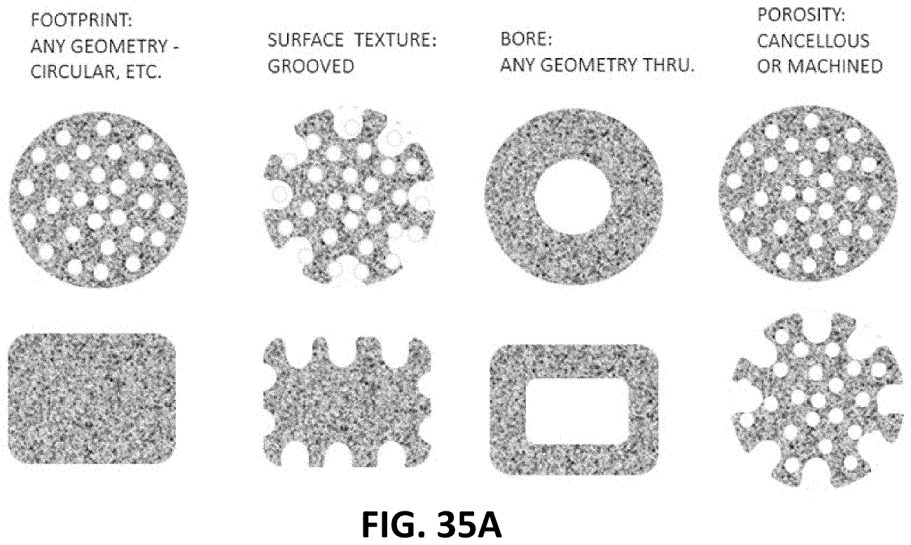

FIGS. 33A and 33B depict perspective and top plan views of one exemplary embodiment of a cage structure 3300 with an insertable/removeable plug 3310 positioned therein. In this embodiment, the plug 3310 is significantly smaller than an opening 3320 within the cage 3300, which allows for the packing of bone graft (not shown) within the opening 3320 when the plug 3310 is positioned therein. In contrast, FIGS. 34A and 34B depict perspective views of another exemplary embodiment of a cage structure 3400 with an insertable/removeable plug 3410 positioned within the cage, where the plug is sized to fit snugly within the opening.

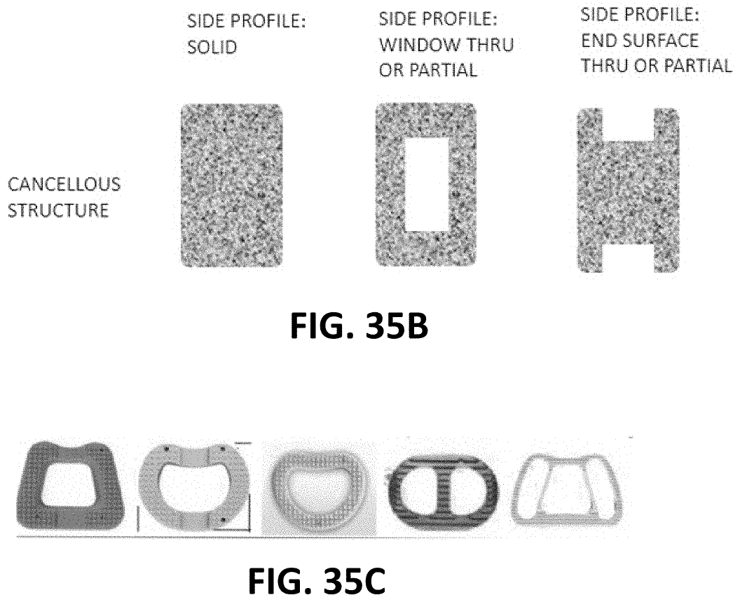

FIG. 35C depicts a top plan view of various commercially available cages, each having a different shape and/or size of the opening and/or openings therein. Desirably, a custom geometry of central plugs can be created to fit each unique cage (either “snugly” or loose to accommodate additional bone graft in the opening, if desired), or a series of different shaped and/or sized plugs can be created and provided in a kit. As depicted in FIGS. 35A and 35B, plugs of various shapes, sized and/or geometries can be created and/or utilized as desired, including plugs incorporating the different surface features described herein.

In various embodiments the disclosed implants will desirably incorporate materials such as silicon nitride that are “phase stable” to a desired degree. For example, Various embodiments will desirably withstand standard autoclave sterilization conditions such as 120° C. 1 atmosphere steam for up to 100 hours of time, with no appreciable change in phase composition, no appreciable change in flexural strength and an inherently stable microstructure. Moreover, such materials will desirably provide favorable imaging characteristics, such as high levels of radiolucency and/or no significant MRI or CT scan artifacts.

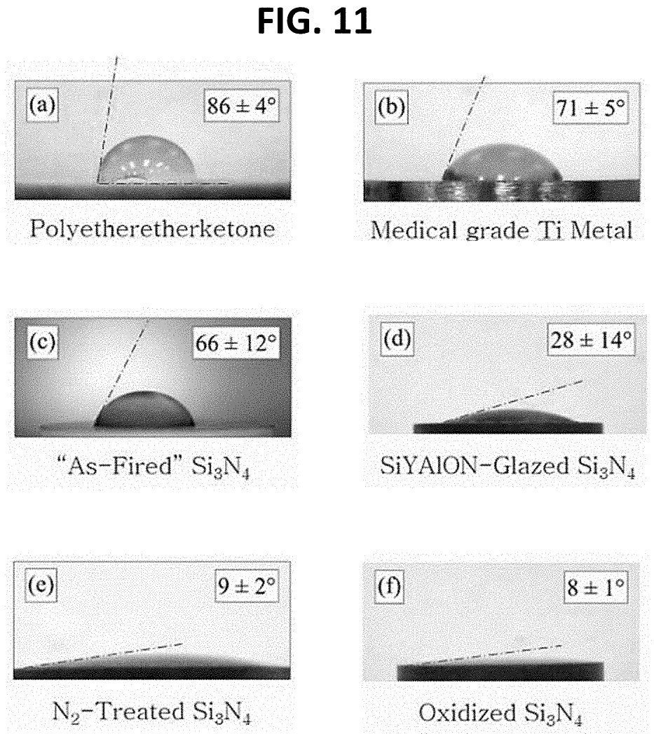

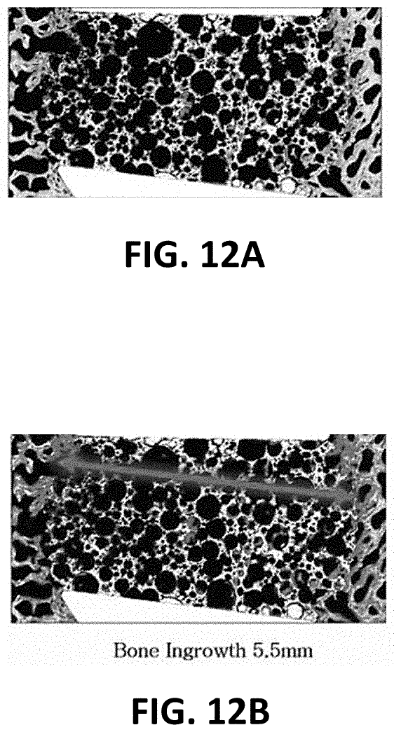

FIG. 11 depicts exemplary degrees of hydrophobicity for various medical grade materials, including silicon nitride in various forms utilized herein. As shown, silicon nitride is much less resistant to water penetration than other materials, which can be a highly desirably characteristic in many applications. In many applications, a porous implant formed from silicon nitride can induce neovascularization within the porous sections of the implant, including internal pores colonized with mineralized bone to a depth exceeding 5.5 mm, such as depicted in FIGS. 12A and 12B.

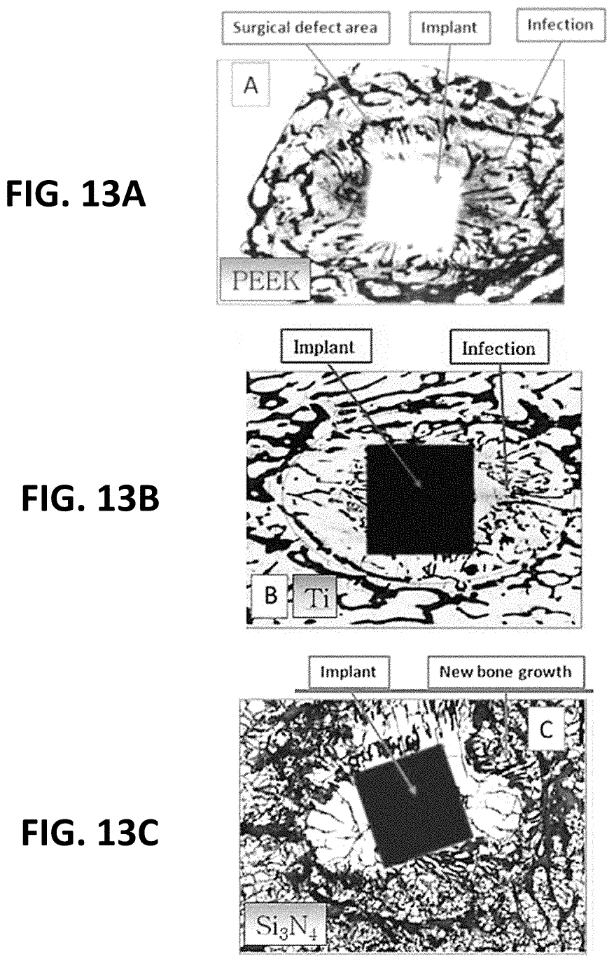

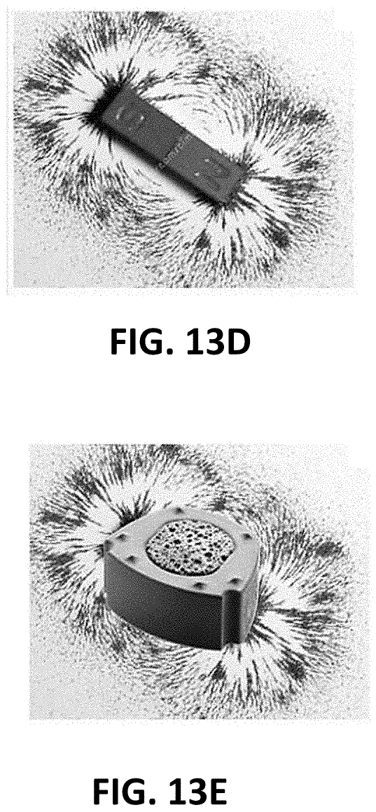

FIGS. 13A through 13C depict three exemplary implants made of PEEK, Titanium and silicon nitride and their effects on adjacent living bone. As shown in FIG. 13A, a PEEK implant may often be accompanied by surgical bone defects that do not fill in with new bone over time, as well as potential infection sites proximate to the implant that may be difficult or impossible to resolve (potentially necessitating implant removal in some cases). In a similar manner, as shown in FIG. 13B, bone infection sites near titanium implants can also be difficult or impossible to resolve, and may similarly necessitate implant removal. However, with a silicon nitride implant, such as shown in FIG. 13C, the surface chemistry of the implant actively destroys infectious bacterial agents, and also induces new bone growth immediately upon implantation. In essence, the effect of the silicon nitride material on new bone growth acts like a magnet on ferrous materials (see FIG. 13D), actively “drawing” new bone near and into the implant (see FIG. 13E).

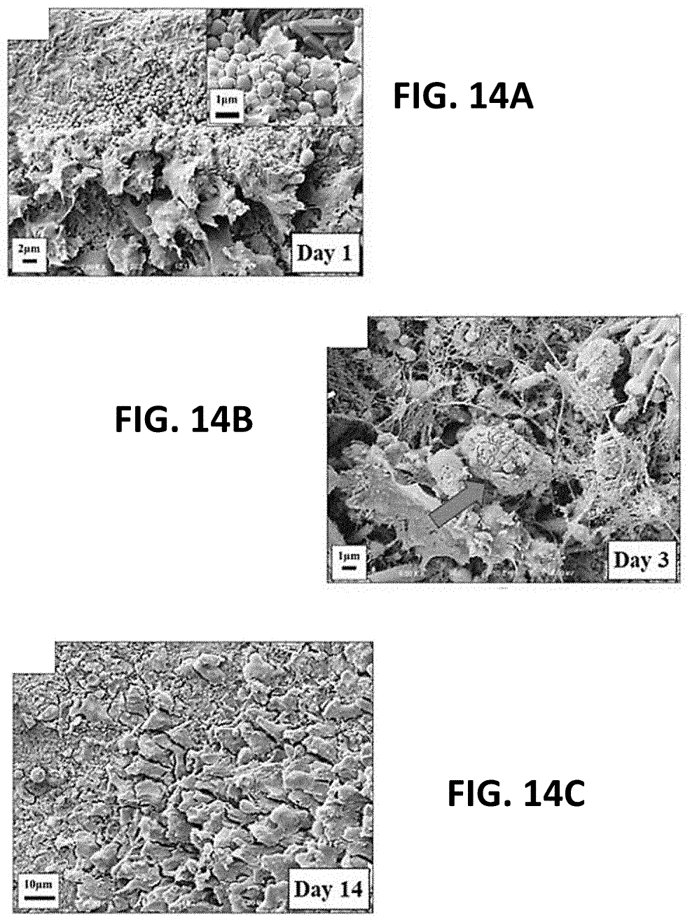

Another significant advantage of using silicon nitride materials in bone implants is the anti-bacterial effects of the material on infectious agents. As best seen in FIG. 14A, upon implantation a silicon nitride surface can induce an inflammatory response action which attacks bacterial biofilms near the implant. This reaction can also induce the elevation of bacterial pods above the implant surface by fibrin cables (see FIG. 14B). Eventually the bacteria in the vicinity of the silicon nitride implant surfaces will be cleared by macrophage action, along with the formation of osteoblastic-like cells (See FIG. 14C). In various experiments involving comparisons between standard implants and silicon nitride implants (both bulk and silicon nitride coated implants of standard materials), cell viability data in (which were determined at exposure times of 24 and 48 hours, showed the existence of a larger population of bacteria on the standard medical materials as compared to Si₃N₄ implants (both coated and bulk). A statistically validated decreasing trend for the bacterial population with time was detected on both coated and bulk substrates, with a highest decrease rate on Si₃N₄-coated substrates. Moreover, the fraction of dead bacteria at 48 h was negligible on the standard implants, while almost the totality of bacteria underwent lysis on the Si₃N₄ substrates. In addition, optical density data provided a direct assessment of the high efficacy of the Si₃N₄ surfaces in reducing bacterial adhesion.

In various embodiments, silicon nitride materials can be incorporated into a variety of implants and implant-like materials, including (1) orthopedic bone fusion implants (i.e., screws, cages, cables, rods, plugs, pins), (2) dental implants, (3) cranial/maxillofacial implants, (4) extremity implants, (5) hip and joint implants, (6) bone cements, powders, putties, gels, foams, meshes, cables, braided elements, and (7) bone anchoring elements and/or features. Where a surface coating of silicon nitride is added to an existing implant, such as to a titanium implant using a 3D-laser-sintering manufacturing process of deposition, this surface coating may comprise a dense, tenaciously adherent Si₃N₄ coating (with thickness 10-20 μm) onto the porous T-alloy surface of commercially available components, which may achieve rapid osseous fixation, while resisting bacteria.

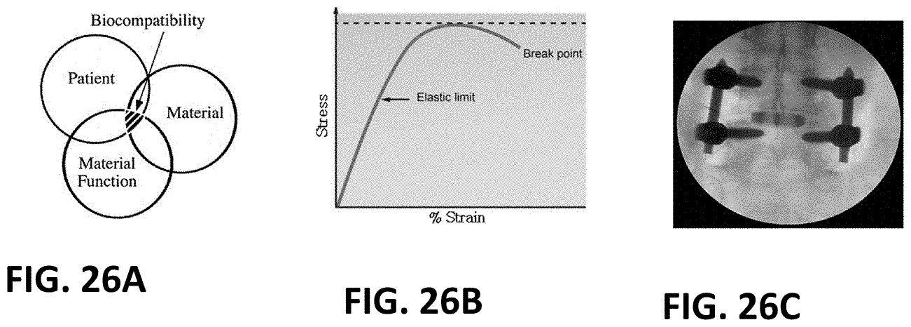

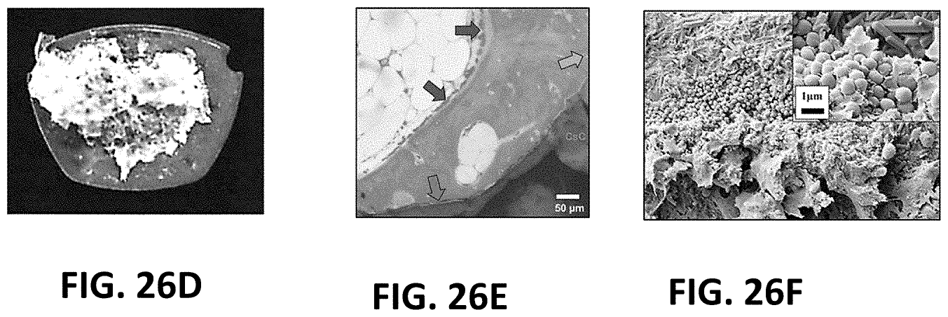

In various embodiments, the disclosed implants can desirably provide various combinations of significant advantages and desirable attributes of an abiotic spinal spacer or similar implant, such as one or more of the following: biocompatibility (FIG. 26A), mechanical integrity (FIG. 26B), radiological traceability (FIG. 26C), osteoconductivity (FIG. 26D), osteoinductivity (FIG. 26E) and/or bacteriostasis (FIG. 26F).

Because many forms of silicon nitride exhibit ceramic-like mechanical properties, these materials may not be well suited for use in screws that may be more than 4 mm in diameter and 15 mm in length, which can be subject to various brittleness failures when inserted into a bone. For spinal applications, where bigger diameter screws such as up to 10.5 mm in diameter and lengths up to 120 mm long may be required, more traditional implants of metal may be desirous for implantation, such as to overcome friction and hardness of human/animal bone. Thus, a typical screw consisting of a single material, screw head, threaded shaft, and tapered tip with cutting flutes may desirably be reconfigured where the threaded shaft portion is partially made of a bone-growth enhancing non-metallic material such a silicon nitrate, particularly on the surface where it contacts the bone. Various methods to integrate such component can be used, such as making a threaded sleeve of silicon nitrate material. Many methods for assembling such a design can be utilized, such as employing a horseshoe shaped sleeve which engages around a single piece central column of a pedicle screw. In various alternative embodiments, a threaded cannulated sleeve could be provided, with or without external and/or internal threaded features, and even where the base screw head and/or shaft with tip could comprise multiple components and/or multiple materials to make the assembly functional and durable. In some embodiments, a surface of the sleeve component could be configured with patterns and/or textures to further increase the surface area of bone contact within a pre-tapped hole in the bone.

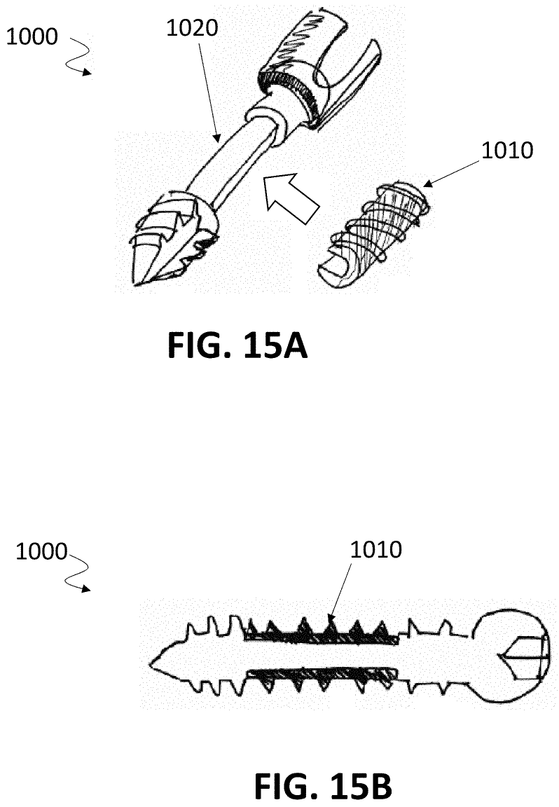

FIGS. 15A and 15B depict another exemplary embodiment of a surgical implant that incorporates silicon nitride features to enhance osseous integration and/or improve bacterial resistance. In this embodiment, a bone growth enhancing screw 1000 is provided, the screw having a screw body 1005 and a modular threaded sleeve 1010 that can be placed onto a central shaft 1020 of the screw body 1005. Desirably, the screw body 1005 will comprise a metallic material such as titanium, which is a commonly accepted and highly tested medical material for bone screws. However, because metal bone screws may not contribute significantly to osseous fixation, the sleeve can comprise a material such as silicon nitride or similar materials that desirably induce osseous integration. Such an arrangement allows silicon nitride to be integrated into the metal bone screw without sacrificing significant strength and/or durability of the screw. Alternatively, a coating of silicon nitride could be applied to one or more surfaces of the bone screw (i.e., through a laser sintering or other method), as previously described. In various alternative embodiments, Si₃N₄ powder may be laser sintered to titanium or PEEK base materials.

In various embodiments, silicon nitride can be manufactured into various shapes and/or sizes, and can be attached to a shaft or other feature of a bone screw as described herein. Because silicon nitride may not be effective on a cutting surface, the cutting tip of the bone screw may desirably comprise a metal cutting tip. Moreover, because the silicon nitride material may shrink or otherwise deform during portions of the manufacturing and/or curing process, it is desirable that the implant design features accommodate potential changes in the design of the insert or similar components. In at least one alternative embodiment, silicon nitride material may be manufactured in a sleeve or other shape, with the corresponding metal screw shape subsequently being modified to accommodate the final cured shape and/or size of the silicon nitride sleeve insert. In various alternative embodiments, the sleeve insert could alternatively comprise a silicon nitride tip or “washer” placed around the screw head, or silicon nitride strips, inserts or “teeth” could be provided along the longitudinal length of the screw.

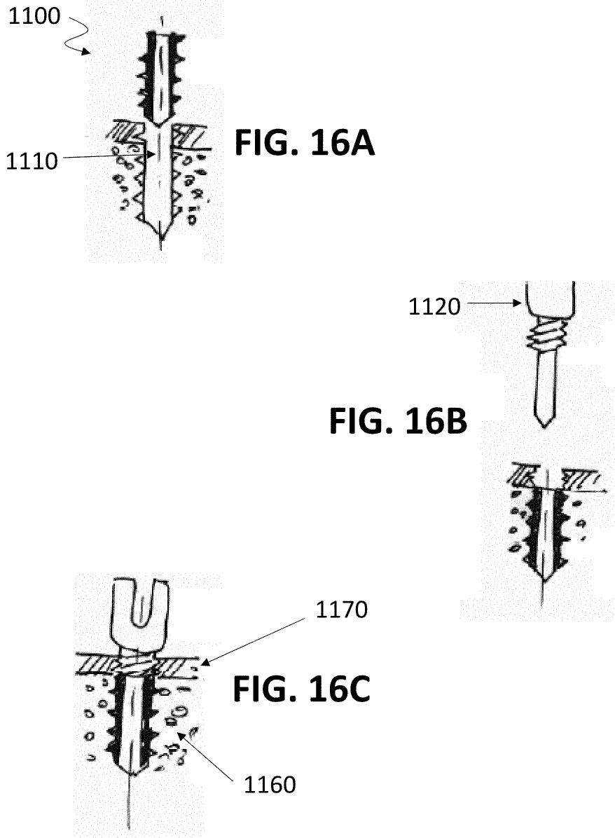

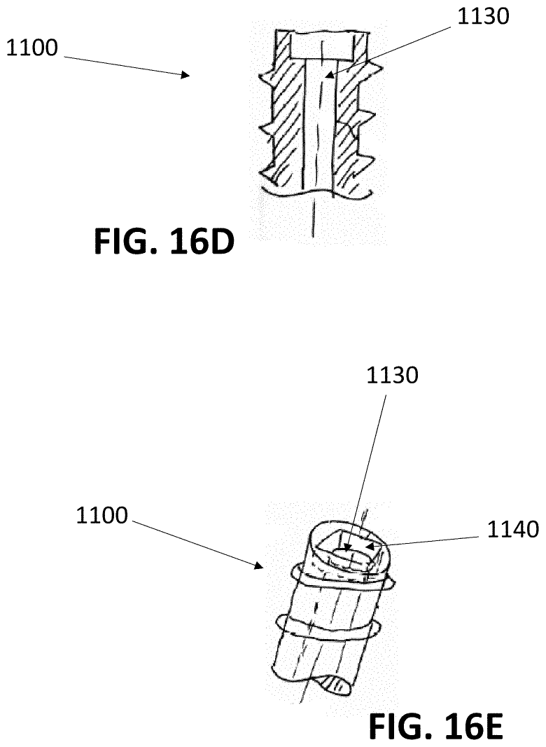

In other embodiments, such as for application to hard bone where the bone may be already tapped, a silicon nitride sleeve element with thread that spans from a screw neck region to a distal tip of a bone screw may be utilized, where the sleeve is inserted into the pre-tapped bore, then the screw body is inserted onto the former and locks axially and/or rotationally. In some embodiment, the sleeve and/or screw body could be pre-assembled and inserted simultaneously into a pre-tapped bore. FIGS. 16A through 16E depict another exemplary embodiment of a surgical implant that incorporates silicon nitride features to enhance osseous integration and/or improve bacterial resistance. In this embodiment, a silicon nitride anchor or sleeve 1100 can be inserted into a prepared hole 1110 in a patient's anatomy, and then a bone screw 1120 or similar device can be inserted into an opening 1130 through the center of the sleeve. If desired, the hole 1110 can be prepared by drilling and/or tapping, and the sleeve 1100 can be advanced into the hole 1110 in a variety of ways. If desired, the sleeve 1100 can include external and/or internal threading, as well as a driving feature 1140 (see FIG. 16E), which can allow the sleeve 1100 to be rotated using a surgical driving tool for advancement into the hole 1110. As best seen in FIG. 16D, one embodiment of a sleeve will desirably provide supplemental fixation to a cancellous region 1160 of the bone, while concurrently allowing a screw thread portion of the screw to engage with a cortical layer 1170 of the bone.

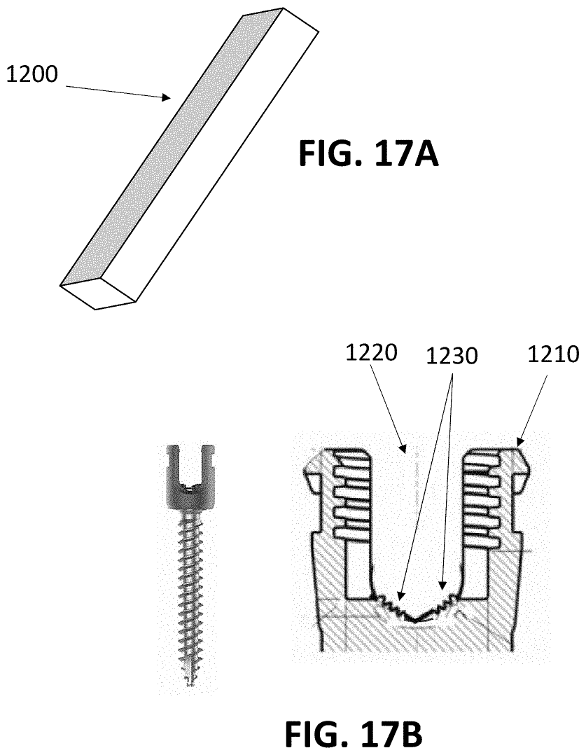

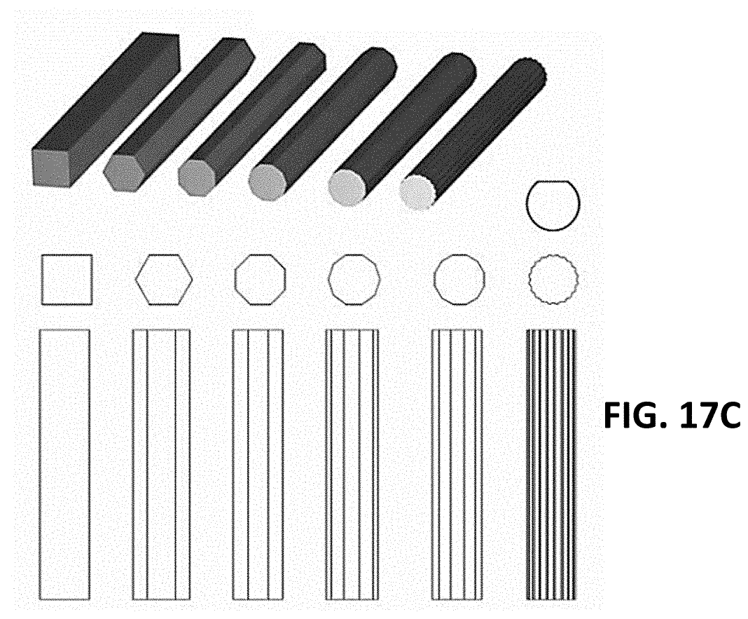

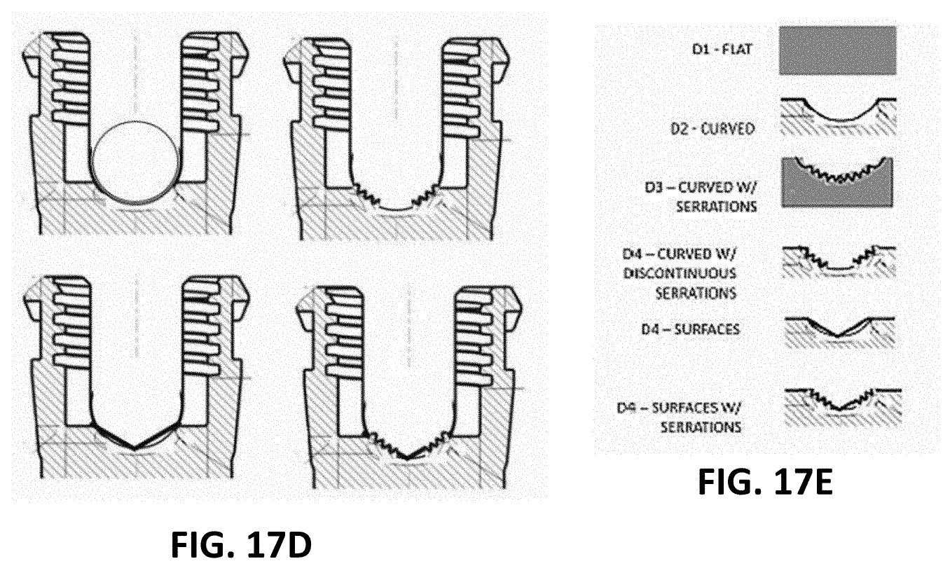

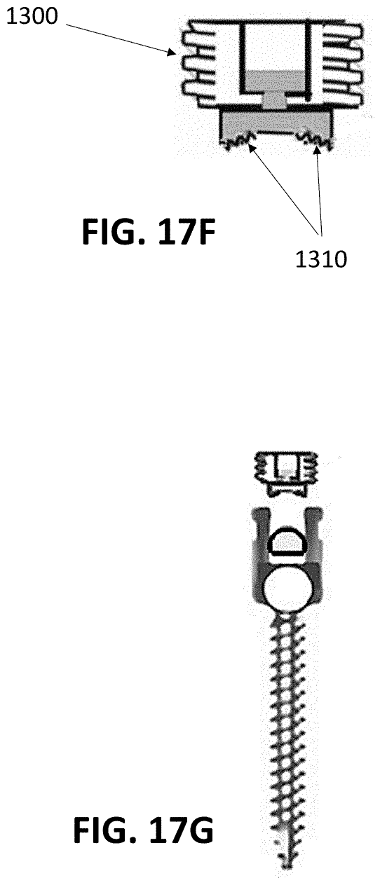

FIG. 17A depicts view of another alternative embodiment of a modular implant component for employment in surgery of the spine and other anatomical regions. In this embodiment, a connecting rod or shaft 1200 (which may optionally comprise silicon nitride in some portion thereof) is disclosed which provides improved fixation and stability to a surgical implant construct, which may be particularly useful in deformity correction and/or various types of spinal surgery. In this embodiment, the connecting rod 1200 desirably includes roughened and/or non-smooth external features, which can include a variety of polygonal shapes such as square and/or hexagonal rods surfaces, which desirably interact with a corresponding screw head such as depicted in FIG. 17B. The screw head 1210 desirably includes a generally U-shaped saddle or receptacle 1220 which includes one or more engagement features 1230 for engaging with external portions of the shaft 1200, desirably inhibiting and/or preventing rotation of the shaft within the screw head. FIG. 17C depicts shaft of varying configurations, FIG. 17D depicts corresponding screw heads of varying configurations, and FIG. 17E depicts various alternative engagement features.

FIG. 17F depicts a cross-sectional view of a tightening screw or set screw 1300, which can be utilized to secure a connecting shaft to a screw head. In this embodiment, the set screw includes one or more engagement features 1310 for engaging with external portions of the shaft 1200, desirably inhibiting and/or preventing rotation of the shaft within the screw head. FIG. 17G depicts an exemplary screw and rod assembly.

The various embodiments herein may optionally encompass bone cement formulations that incorporate silicon nitride (i.e., Si₃N₄ and/or chemical analogues thereof) in their mixtures and/or composition, which may include the incorporation of silicon nitride powders, granules, particulates, portions, pebbles, blocks, layers and/or coatings of solids and/or particulates within the bone cement mixture. In various embodiments, the bone cement may be a liquid, paste, gel or dough, and preferably hardens to a substantially solid solidified material.

In at least one exemplary embodiment, a PMMA bone cement formulation comprising a powered MMA-styrene co-polymer and a reactive monomer such as methyl methacrylate can be combined with various percentages by weight and/or volume of a ceramic material such as a silicon nitride material, which when mixed and polymerized can result in a polymerized and/or “cured” block, implant and/or structure capable of implantation in a bony defect and/or other location. In various embodiments, the ceramic material may comprise a granular or regularly/irregularly shaped material, with the granules having a plurality of interconnecting micropores. In various embodiments, a plurality of different sizes of granules may be used.

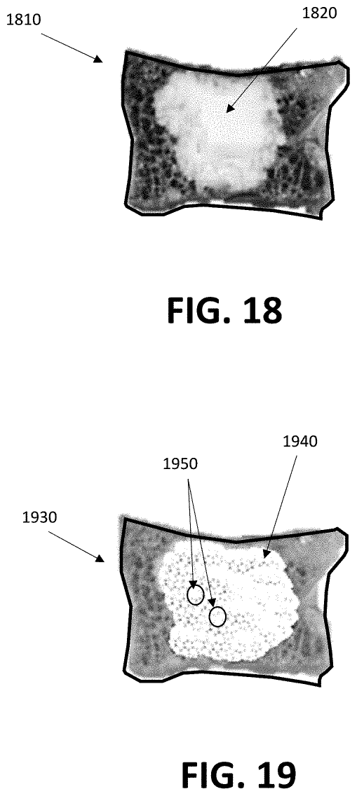

Historically, PMMA has been established as a very most material for fixation in joint replacement surgery. Polymerization of methyl methacrylate is a reaction that results in a doughy substance that self-cures in a short time. PMMA is made of a methyl methacrylate monomer precursor that polymerizes to form PMMA. There are a number of commercially manufactured PMMA cements available, each cement kit comprising an individually packaged granules and a liquid. The package typically comprises a powdered PMMA as its major constituent, together with a liquid vial which contains the monomer sub-unit, methyl methacrylate. Additionally, there are a number of other chemicals included to start and regulate the polymerization process (such as benzoyl peroxide). Additionally, opacifiers or oligomers of PMMA may also be contained. FIG. 18 depicts an exemplary vertebral bone 1810 filled with a PMMA bone cement 1820.

In at least one example, a PMMA bone cement formulation comprising a powered MMA-styrene co-polymer and a reactive monomer such as methyl methacrylate can be combined with various percentages by weight and/or volume of a powdered, granulated and/or fluidized silicon nitride material, which when mixed can create a flowable and/or moldable material which will desirably harden and/or polymerize into a variety of shapes, which can include injection of the flowable material through a syringe or tube into a void or opening to partially and/or fully fill the void or opening, wherein the material will subsequently harden and/or polymerize into a shape which can be defined by the cavity in which it sits. This could include the injection into various anatomical locations as well as injection and/or introduction into implants and/or other devices prior to, during and/or after their implantation into a targeted patient anatomy (i.e., such as within the graft chamber of an intervertebral fusion implant). As best seen in FIG. 19, this case include the injection of a PMMA/silicon nitride cement bolus 1940 into a vertebra body 1930, with the cement bolus 1940 including a plurality of silicon nitride granules 1950 therein.

In various embodiments, a settable bone cement of similar material could comprise a PMMA or other type of bone cement, in combination with silicon nitride and/or a resorbable granular material such as calcium phosphate or other material, which facilitates bone ingrowth, bone outgrowth and/or bone through-growth in varying amounts. Such a cement could provide the improved bacteriostatic properties of silicon nitride, and also allow for superior adhesions and/or anchoring of the cement to surrounding structures. Unlike typical bone cements, which may not interdigitate and/or which may be a source for bacterial infection, cements of the present invention inhibit and/or prevent the presence of bacteria within the bone cement bed. Moreover, unlike antibiotic-loaded bone cements, the bacteriostatic properties of silicon nitride are not anticipated to appreciably fade or diminish over time, and the present of silicon nitride within the cement mixture does not markedly weaken the strength and/or durability of the cured and/or polymerized cement.

In accordance with various aspects of the present subject matter, bone cements and/or other implants, devices and/or components thereof are described that incorporate silicon nitride (i.e., Si3N4 and/or chemical analogues thereof) in their construction, either in the entirety of the implant as well as components, portions, layers and/or surfaces thereof. In various embodiments, the silicon nitride material(s) will be highly osteo-inductive and/or osteoconductive and will desirably facilitate and/or promote fixation to adjacent living bone surfaces, while concurrently reducing and/or inhibiting periprosthetic infection and/or bacterial adhesion.

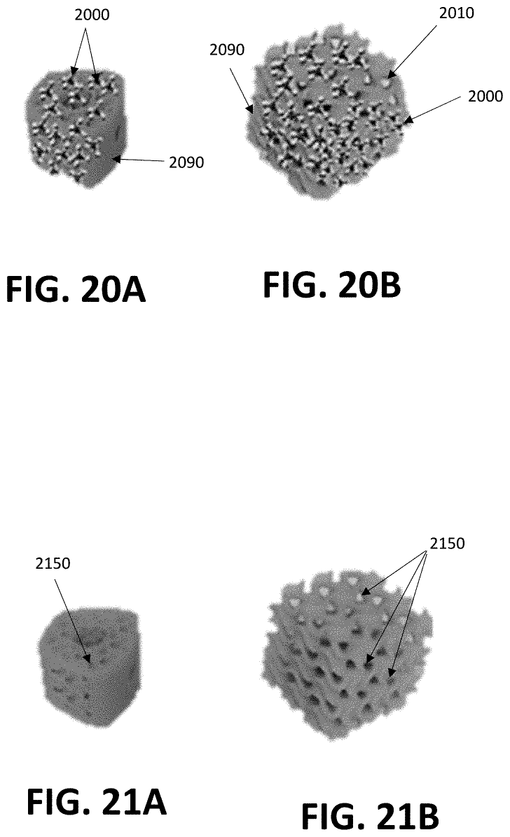

As best seen in FIGS. 20A and 20B, a settable silicon nitride cement may desirably comprise a PMMA base 2090 with a plurality of silicon nitride granules 2000 therein. In various embodiments, the granules can be mixed with and suspended within the curing and/or cured PMMA, with various portions of the silicon nitride granules exposed to the surrounding anatomy. In various embodiments, such as shown in FIG. 20B, the PMMA base 2090 can further optionally include openings and/or voids 2010 formed therein. FIGS. 21A and 21B depict the settable cement blocks of FIGS. 20A and 20B after partial resorption of silicon nitride granules (or other resorbable material granules) near the surface of the blocks, wherein additional macro pores 2150 have been formed as some of the silicon nitride granules/resorbable material granules have been resorbed and/or remodeled by the patient.

In various applications, the utility of silicon nitride as a component of medical implants may be further enhanced by the addition of various other medical materials, including the use of one or various combinations of titanium, chrome cobalt, stainless steel, silicone, poly (ether ether ketone) (PEEK), ultra-high molecular-weight polyethylene (UHMWPE), polyurethane foams, polylactic acid, apatites and/or various 3D printed materials. In such cases, the employment of such material mixtures in implant construction may enhance the strength and/or durability of a desired implant design, as well as allow for improved surgical outcomes and/or greatly reduced complication rates.

The various cements, mixtures, devices, implants and/or components thereof disclosed herein can incorporate a silicon nitride material (i.e., Si3N4 and/or chemical analogues thereof) in their construction, either in one or more of a two part mixture, as well as within the entirety of an implant as well as components, portions, layers and/or surfaces thereof. The incorporation of silicon nitride as a component material for spinal implants can provide significant improvements over existing implant materials and material designs currently available, as the silicon nitride material(s) will be highly osteo-inductive and/or osteoconductive and will desirably facilitate and/or promote implant fixation to adjacent living bone surfaces, while concurrently reducing and/or inhibiting periprosthetic infection and/or bacterial adhesion to the surfaces and/or interior portions thereof.

Silicon nitride (Si3N4) and its various analogs can impart both antibacterial and osteogenic properties to an implant, including to mixtures containing Si2N4 and/or bulk Si3N4 as well as to implants coated with layers of Si3N4 of varying thicknesses. In bone replacement as well as prosthetic joint fusion and/or replacement, osseous fixation of implants through direct bone ingrowth (i.e., cementless fixation) is often preferred, and such is often attempted using various surface treatments and/or the incorporation of porous surface layers (i.e., porous Ti6Al4V alloy) on one or more bone-facing surfaces of an implant. Silicon nitride surfaces and/or interior portions express reactive nitrogen species (RNS) that promote cell differentiation and osteogenesis, while resisting both gram-positive and gram-negative bacteria. This dual advantage of RNS in terms of promoting osteogenesis, while discouraging bacterial proliferation, can be of significant utility in a variety of implant designs.

Desirably, the inclusion of silicon nitride components into a given cement mixture will encompass the use of granularized and/or powdered silicon nitride, as well as bulk silicon nitride, as well as implants incorporating other materials that may also include silicon nitride components and/or layers therein, with the silicon nitride becoming an active agent of bone fusion. RNS such as N2O, NO, and —OONO are highly effective biocidal agents, and the unique surface chemistries of Si3N4 facilitate its activity as an exogenous NO donor. Spontaneous RNS elution from Si3N4 discourages surface bacterial adhesion and activity, and unlike other direct eluting sources of exogenous NO, Si3N4 elutes mainly NH4+ and a small fraction of NH3 ions at physiological pH, because of surface hydrolysis and homolytic cleavage of the Si—N covalent bond. Ammonium NH4+ can enter the cytoplasmic space of cells in controlled concentrations and through specific transporters, and is a nutrient used by cells to synthesize building-block proteins for enzymes and genetic compounds, thus sustaining cell differentiation and proliferation. Together with the leaching of orthosilicic acid and related compounds, NH4+ promotes osteoblast synthesis of bone tissue and stimulates collagen type 1 synthesis in human osteoblasts. Conversely, highly volatile ammonia NH3 can freely penetrate the external membrane and directly target the stability of DNA/RNA structures in bacterial cells. However, the release of unpaired electrons from the mitochondria in eukaryotic cells activates a cascade of consecutive reactions, which starts with NH3 oxidation into hydroxylamine NH₂OH (ammonia monooxygenase) along with an additional reductant contribution leading to further oxidation into NO2- nitrite through a process of hydroxylamine oxidoreductase. This latter process involves nitric oxide NO formation. In Si3N4, the elution kinetics of such nitrogen species is slow but continuous, thus providing long-term efficacy against bacterial colonies including mutants (which, unlike eukaryotic cells, lack mitochondria). However, when slowly delivered, NO radicals have been shown to act in an efficient signaling pathway leading to enhanced differentiation and osteogenic activity of human osteoblasts. Desirably, Si3N4 materials can confer resistance against adhesion of both Gram-positive and Gram-negative bacteria, while stimulating osteoblasts to deposit more bone tissue, and of higher quality.

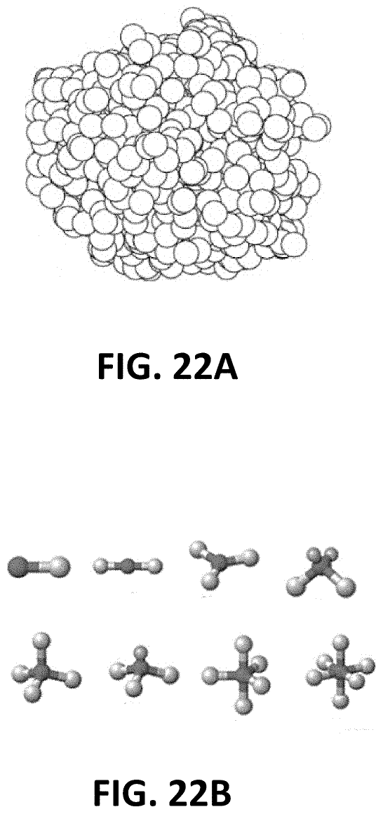

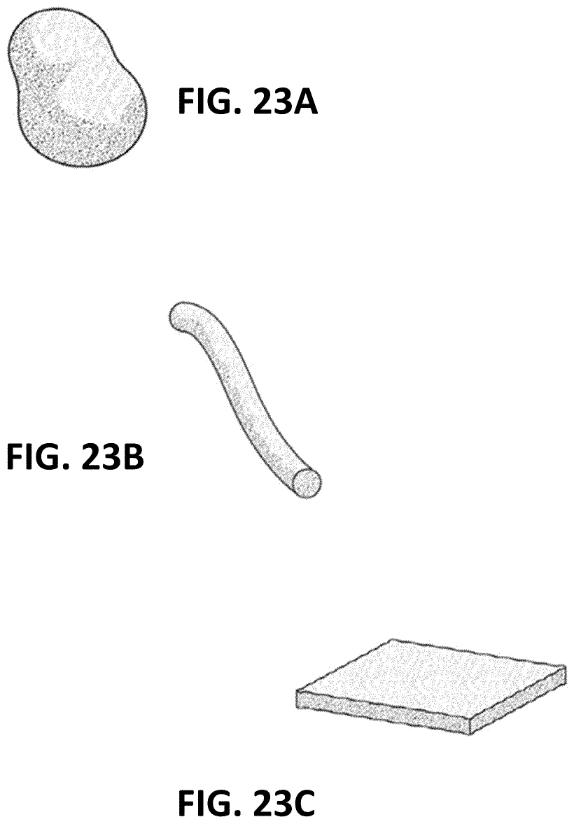

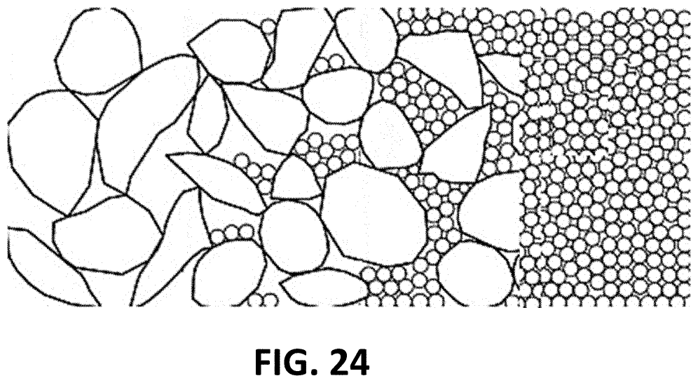

In another exemplary embodiment, disclosed is a bone cement which has both improved structural properties and improved osteoconductivity to regenerate and heal the host bone tissue. In this embodiment, the distribution of a granulated microporous ceramic material such as silicon nitride within bone cement will desirably provide improved structural properties for the hardened bone cement, whilst the microporous structure of the ceramic material granules allows host tissue to bind and regenerate around and within the bone cement-ceramic material mixture. In some embodiments the ceramic material granules may comprise a single average size granule or granule distribution (See FIG. 22A), while in other embodiment, the granule size may be widely distributed and/or essentially random within a range of sizes (See FIG. 24). In another alternative embodiment, at least two different preselected sizes, or ranges of sizes, of granulated material can be used, e.g. in a similar manner to sand and gravel being used with cement to make concrete. The different size of the silicon nitride “sand and gravel” may be helpful in improving the strength of the material. Preferably, the ceramic material will be generally evenly distributed throughout a cross-section of the hardened bone cement, that is substantially without clumps of ceramic material forming. If desired, the various silicon nitride granules may comprise a variety of different shapes, including rounded particles, irregularly shaped particles (see FIG. 23A), elongated particles, fibers or “strings” (see FIG. 23B), flattened or planar particles (see FIG. 23C), or other shapes, or any combination thereof. In many cases, multiple shapes and/or sizes of particles may provide for optimized packing and/or density of the silicon nitride material for certain applications.

If desired, a variety of sizes and/or shapes of silicon nitride granules and/or particles may be utilized in various embodiments of the present invention, which can include particles and/or microparticles that form a variety of geometric bonds and/or matrix shapes, including linear, trigonal planar, bent or angular, tetrahedral, trigonal pyramidal, trigonal bipyramidal, octahedral, and/or other shapes, including those depicted in FIG. 22B.

In various embodiments, the individual granules of the silicon nitride material may have micropores. Preferably, the micropores are interconnecting. They are preferably not confined to the surface of the granules but are found substantially throughout the cross-section of the granules. Preferably, the diameter of the granule particles is between 10 μm and 1 mm, preferably 400 μm and 1000 μm, especially 500-900 μm, 500-800 μm or 600-700 μm. The ceramic material granules may be formed from a fused block of biomaterial by milling or grinding using, for example, a ball mill, and the size of the granules may be adjusted using, e.g. one or more sieves. In this manner, two or more different sized particles, or ranges of sizes of particles, may be obtained from a single “run” of the ball mill, if desired.

Where the silicon nitride granule size within a given cement formulation is distributed between two different preselected sizes, or ranges of sizes, some embodiments may comprise a mixture of small and large granules. For example, the small granules may have a size range of 10 μm to 500 μm, especially 50 to 350 μm, most preferably 100 to 250 μm diameter, while the large granules may have a diameter of 250 μm to 1.5 mm, especially 500 μm to 1 mm, most preferably 600 μm to 800 μm.

In various embodiments, the bone cement may comprise a mixture of a PMMA bone cement or a bone cement precursor with the ceramic material granules. That is, in the solidified bone substitute, the bone cement forms a matrix that binds together the ceramic material granules, in a similar manner to the lime or cement constituting the cementing material that binds together the sand and aggregate in a mortar or concrete. The term “bone cement precursor” is intended to mean one or more compounds which, upon curing or solidifying, form a substantially solid bone cement matrix. For example, with PMMA, methyl methacrylate monomer is polymerized to form the PMMA bone cement. The monomer is a bone cement precursor. Similarly, to form inorganic materials, such as calcium phosphate, bone cement may be formed, for example, by mixing dicalcium phosphate dihydrate with tetracalcium phosphate. These two compounds can act as precursors to the final bone cement. Upon wetting, the two materials react and solidify to form a solidified bone cement. In at least one exemplary embodiment, the bone cement, which may be made from the bone cement precursor, is a polymeric material or an inorganic ceramic material. Preferably, the organic material is a poly (meth) acrylate material, such as PMMA or PAA (polyacrylic acid).

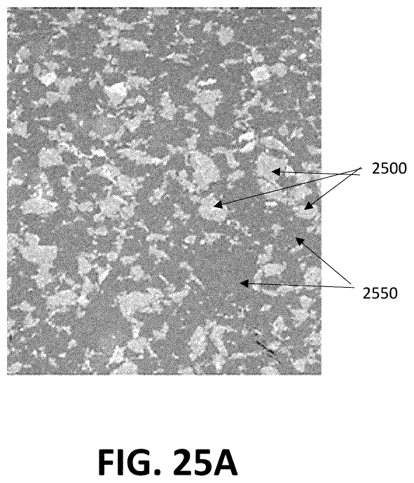

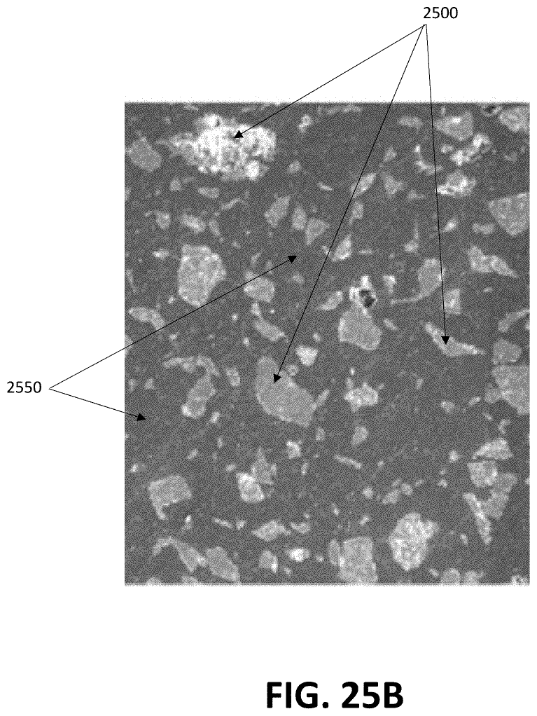

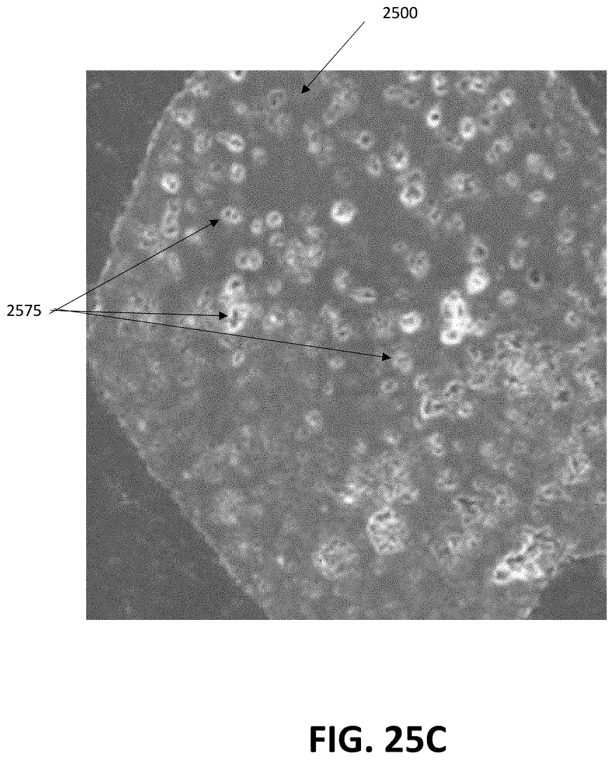

FIGS. 25A and 25B depicts scanning electron microscope (SEM) pictures of an exemplary PMMA/ceramic cement. In this embodiment the ceramic can comprise granules of a silicon nitride material 2500, which is adhered and/or held within a cured PMMA matrix 2550. Where the bone cement material is desirably injectable through a smaller diameter opening and/or incision, the use of bulk and/or large granules of silicon nitride (or other material) implants may not be desirous and/or may be impractical. In such embodiments, it may be desirable to incorporate silicon nitride in a formable and/or curable form, which may include the incorporation of silicon nitride with other materials such as curable bone cements. In such a case it may be desirable for the silicon nitride to be provided in small granular and/or powdered form, to allow easy mixing with the bone cement constituents. Desirably, the size of the granules allows it to be injected through the bore of a needle from, for example, a syringe, into position on the surface of a bone under repair. For example, the preferable maximum size of silicon nitride granules and/or particles in such an application may be 0.1 mm, or 0.5 mm, or 1.0 mm, or 1.5 mm.

As best seen in FIG. 25C, the silicon nitride granules 2500 can each include a plurality of micropores 2575 formed therein, with the micropores of varying shapes and/or sized within an individual granule. In various aspects of the invention, the ceramic granules will each include a plurality of micropores formed therein. In some aspects, the invention provides: a bone substitute comprising a mixture of a bone cement or a bone cement precursor and silicon nitride material granules, the silicon nitride granules having a plurality of micropores of an average diameter of between 1 μm and 10 μm and/or between 10 μm and 50 μm and/or between 50 μm and 100 μm. Of course, smaller and/or larger pore sizes within the granules may have particular utility in certain applications.

In various embodiments, the PMMA described herein may be replaced by bisphenol-alpha-glycidyl methacrylate resin (BIS-GNA) to form alternative organic bone cements. Alternatively, PAA may be used in combination with aluminosilicate glass to form silicate cement or “glass ionomer cement” (GIC).

If desired, the bone cement mixtures described herein may additionally comprise one or more additional materials such as accelerators or regulators in order to control the curing of the bone cement. Catalysts of organic polymerization reactions include peroxides, such as benzyl peroxide. Accelerators of inorganic cements are known, for example disodium hydrogen phosphate is known to be used as an accelerator. Opacifiers or colorants may also be included. Additionally, polystyrene may be included as necessary to improve the handling of the properties of the cement.

In various embodiments, the volume and/or weight ratio of silicon nitride to cement may be 1000:1, 100:1, 50:1, 10:1, 5:1, 2:1, 1:1, 1:2, 1:5, 1:10, 1:50. 1:100 and/or 1:1000 or lesser/greater, and/or any ranges between any combination of the above.

If desired, the bone cement mixture may additionally comprise one or more pharmaceutically and/or biologically active compounds. These may be incorporated into the micropores and/or mid-pores and in use may be used to stimulate cell growth around and into the biomaterial. For example, growth factors, such as transforming growth factor (TGF-βI), bone morphogenetic protein (BMP-2) or osteogenic protein (OP-I) maybe incorporated into the biomaterial. Further materials such as enzymes, vitamins (including Vitamin D) and trace material such as zinc (for example in the form of salt) may also be incorporated.

The ceramic material used to produce the granules may be any non-toxic ceramic known in the art, but in various embodiments will comprise a silicon nitride material and/or its chemical analogues.

In another exemplary embodiment, the PMMA may desirably be located primarily between the granules of silicon nitride, such that the PMMA adheres the adjacent granules together without completely encompassing each of the granules. In such a case, the resulting silicon nitride “block” or implant may have a spongy or swiss-cheese-like appearance. In this embodiment, a liquid monomer may be distributed within some of the pores of the silicon nitride granules, with powdered PMMA later mixed with and/or “dusted over” the granules to cause polymerization of the PMMA between the silicon nitride granules (i.e., similar to a “gluing” agent). Coating of the silicon nitride particles in this manner may improve the distribution of the particles through the finely fused product and produce a substantially uniform product with substantially evenly distributed micropores. Where the coating agent is liquid, for example PEG, simply mixing the ceramic particles in the coating agent may coat the particles. Alternatively, some coating agents, such as the starch and agar coating agents may be mixed with an inert liquid, such as water, in a granules form, and heated to allow the starch or agar to form a polymer coating around the particles. Heating liquids containing starch can cause the starch to polymerize and thicken the liquid in a similar manner to adding corn flour to thicken gravy when cooking.

In various embodiments, disclosed are biomaterials obtainable by various manufacturing processes. Bone implants, dental implants or ear, nose or throat (ENT) implants comprising both substitute materials according to the invention are also provided. Additionally, the invention provides the use of the bone substitute as a bone replacement, in dental implants or maxillofacial repair materials for the repair of bone breaks or fractures, osteoporotic bone, intervertebral space implants, and/or as a bone glue or putty or a load bearing surface on a bone. Furthermore, the bone substitute may be used, for example, to create attachment points for devices such as screws or plates.

In various embodiments, a bone cement material can be made by mixing a bone cement precursor, as defined above, together with the silicon nitride material, as defined above, to form a paste and the bone precursor is caused to convert to a substantially solid bone substitute material comprising a bone cement and a ceramic material by, for example, the use of a catalyst or the reaction of the bone precursor materials. While PMMA may be a preferred material in many embodiments, the disclosed inventions similarly contemplate the use of other bone cement materials instead of PMMA. The bone cement precursors may be obtained from manufacturers and used according to the manufacturers' instructions, with the addition of the silicon nitride material granules which is mixed and dispersed within the bone cement prior to its setting.

In one exemplary embodiment, a silicon nitride material can be pulverized, for example using a ball mill or other milling machinery. The size of the resulting silicon nitride granules may be adjusted, for example, by sieving through a mesh of the desired size to regulate the size of the granules. The granules can then be mixed with a bone cement precursor, in a similar manner to adding aggregate to cement to form concrete. The cement precursor may be PMMA, which can be purchased from a variety of well-known manufacturers. Cement kits usually consist of an individually packed granules and a liquid which is typically sterilized by gamma irradiation and ultra-filtration. The packaged granules can contain PMMA as its major constituent. The liquid contains the monomer sub-unit, methyl-methacrylate. Additionally, one or more other ingredients may include chemicals that are responsible for the polymerization reaction rate, as well as the handling properties of the cement and the resistance to degradation. An initiator polymerization, such as benzoyl peroxide, may be provided to start the polymerization reaction. Additionally, polystyrene may be included as this improved the handling of the properties of the cement.

In the exemplary embodiment, a weight ratio of precursor to the silicon nitride material granules may be between 100:1 to 1:100, especially 10:1 to 1:10, and more preferably 1:1.

In various embodiments the silicon nitride granules and/or cement components thereof can be formed using a variety of techniques, including by compressing, milling and firing silicon nitride powder, as well as by extruding silicon nitride into sheet, tube, pipe and/or thread form (which may be further processed into thread or “rope” by braiding and/or other techniques). Silicon nitride shapes may also be manufactured using subtractive manufacturing techniques (i.e., machining, milling and/or surface roughening), as well as by using additive manufacturing techniques (i.e., surface coating, brazing, welding, bonding, deposition on various material surfaces and/or even by 3D laser printing of structures). If desired, silicon nitride may even be formed using curing or other light/energy activation techniques, such as where a slurry of liquid polymer and silicon nitride particles may be UV cured to create a 3-dimensional structure and/or layer containing silicon nitride. In various embodiments, silicone nitride may be utilized in block form, in sheets, columns and bars, in cable or braided form, in mesh form, in a textured surface coating, in powder form, in granular form, in gel, in putty, in foams and/or as a surface filler and/or coating. In some cases, a surface layer of silicon nitride cement may be formed, placed and/or deposited on an external and/or internal surface of an implant.

Once implanted in a desired location, the silicon nitride cement will desirably be highly osteo-inductive and/or osteoconductive and will desirably facilitate and/or promote fixation of the cement and/or any implants used therewith to adjacent living bone surfaces, while concurrently reducing and/or inhibiting periprosthetic infection and/or bacterial adhesion to the surfaces and/or interior portions of the cement.

If desired, a bone implant could be constructed from a variety of modular components, including at least one “modular” component comprising a silicon nitride material or cement. If desired, such an implant and/or the components thereof could be provided in a kit form for selection and/or assembly in a surgical theatre and/or in situ during a surgical procedure. If desired, various components may be removable and replaceable. If desired, the silicon nitride cement could be provided in a fully cured form, such as in part of the implant, or could be provided in a mixable and/or flowable form, wherein the cement can cure during the surgical procedures.

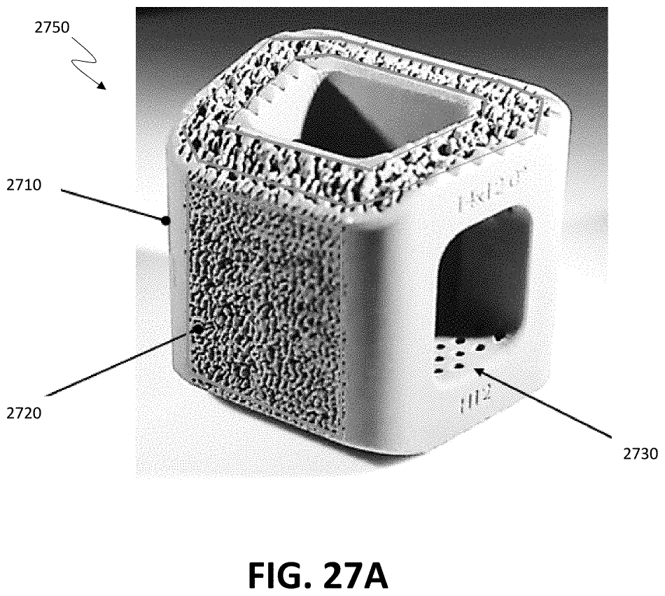

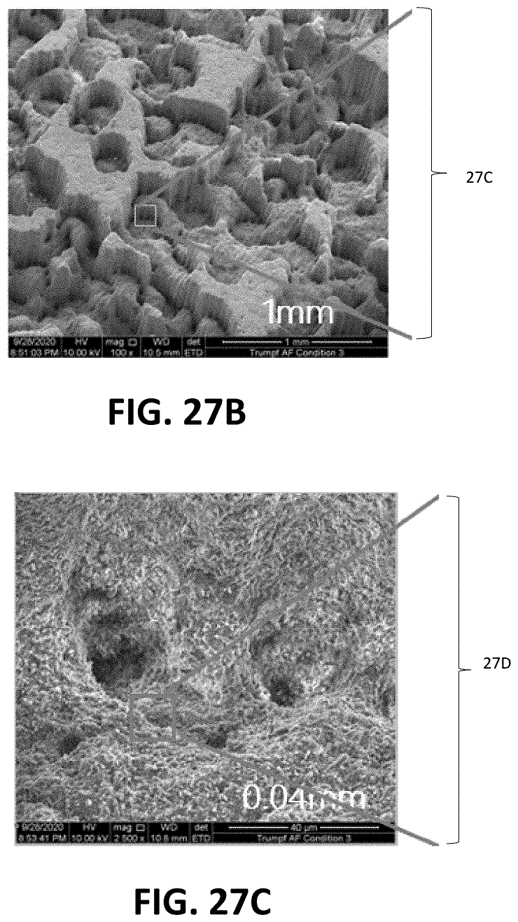

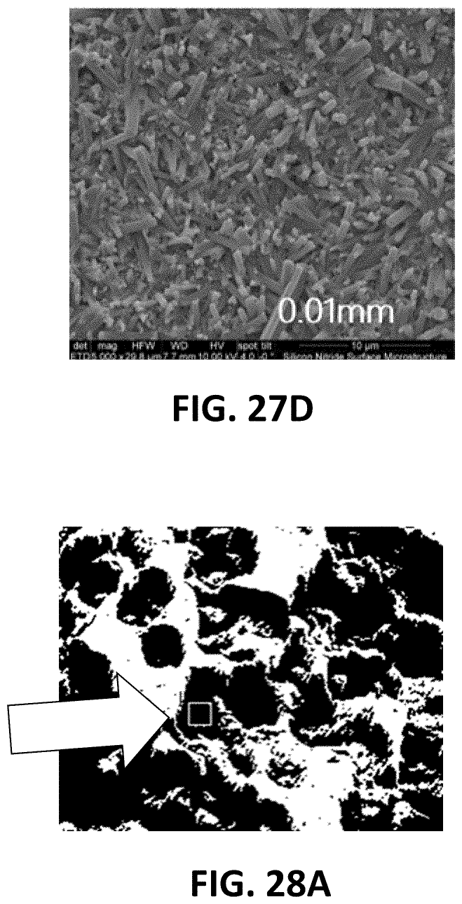

FIG. 27A depicts another exemplary embodiment of a cage structure 2700, wherein the cage can comprise a variety of materials (i.e., one or more of the materials disclosed herein), including being formed from a PEEK of silicon nitride material. In this embodiment, the cage 2700 can include a variety of differing surface features to desirably promote bony ingrowth, such as a visually detectable smooth surface 2710 (which desirably promotes micro-anchoring—as depicted in FIG. 27D), a visually detectable rough surface 2720 (which desirably provides larger voids and/or areas for macro bone anchoring as well as micro-anchoring—as depicted in FIGS. 27B and 27C). Moreover, the cage 2700 may optionally include machined openings such as through and/or partial depth holes 2730 (which may be similar in design and/or function to through-holes depicted in FIGS. 6B and 7A). Desirably, the combination of different surface features within a single implant or components thereof can result in better bony integration and/or bone ingrowth.

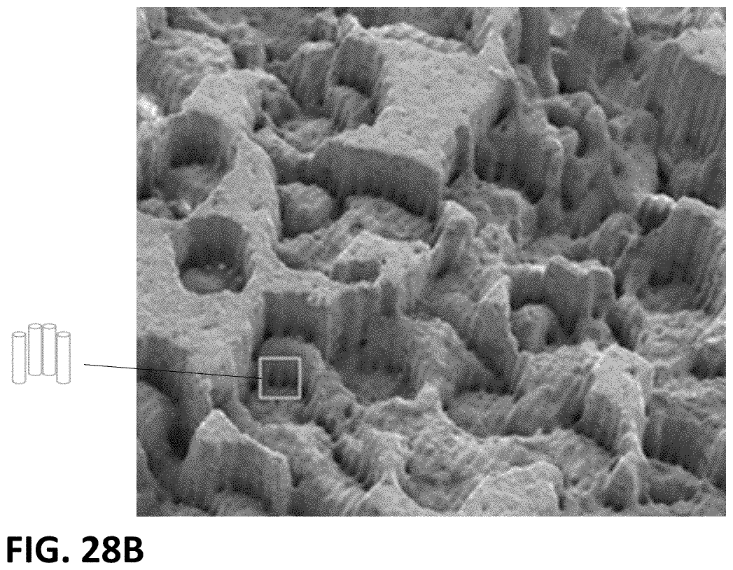

FIG. 28A depicts an external surface of an exemplary implant wherein white indicates peaks while black indicates valleys in the surface texture. The highlighted section of the image of FIG. 28A is depicted in FIG. 28B. In this embodiment, many of the surface features are formed from multiple laser beam passes over the implant surface, wherein each pass desirably creates a cylindrical bore. Such laser beam texturing of implant surfaces can cause melting and vaporization of material which takes place due to ablation when a high-energy beam of laser impinges the work surface. Depending upon the type of implant material used, the material may absorb the laser energy, and this energy is converted into heat, and/or the material may start to melt and vaporize in case of a pyrolytic process. In a photolytic process, absorption of photon may induce chemical reactions which overcome the binding energy of the material. Desirably, a surface texture will be formed that influences osteoblast proliferation, gene expression and/or morphology, which in various embodiments can include surface features of 1 mm or greater in height and/or depth.

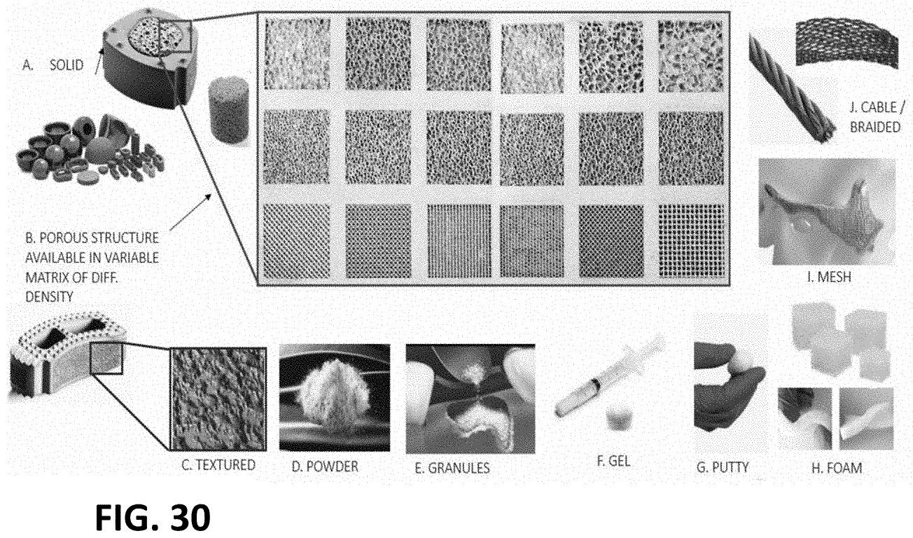

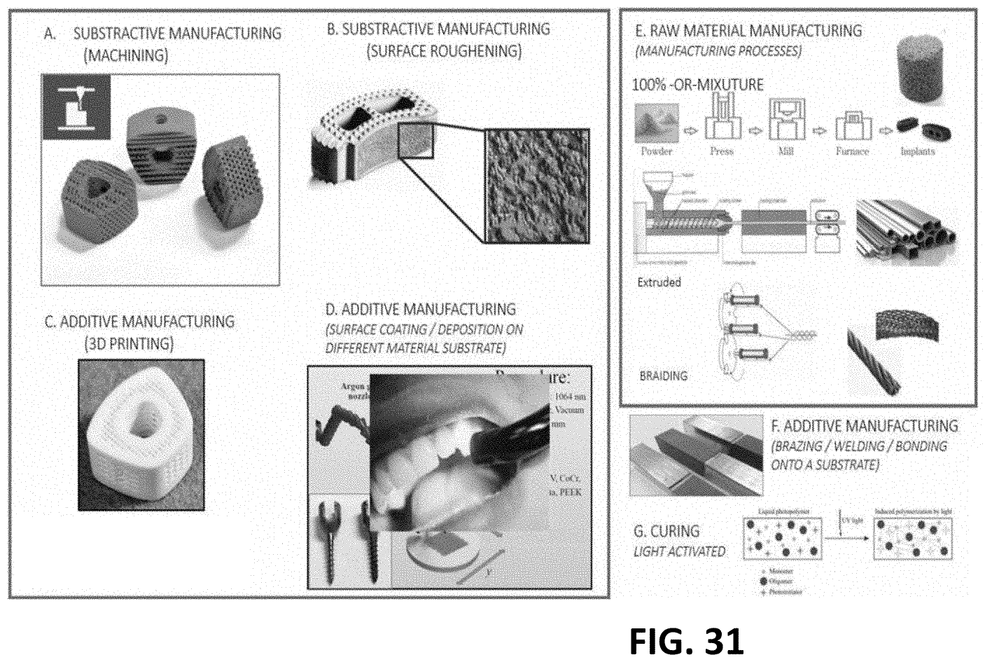

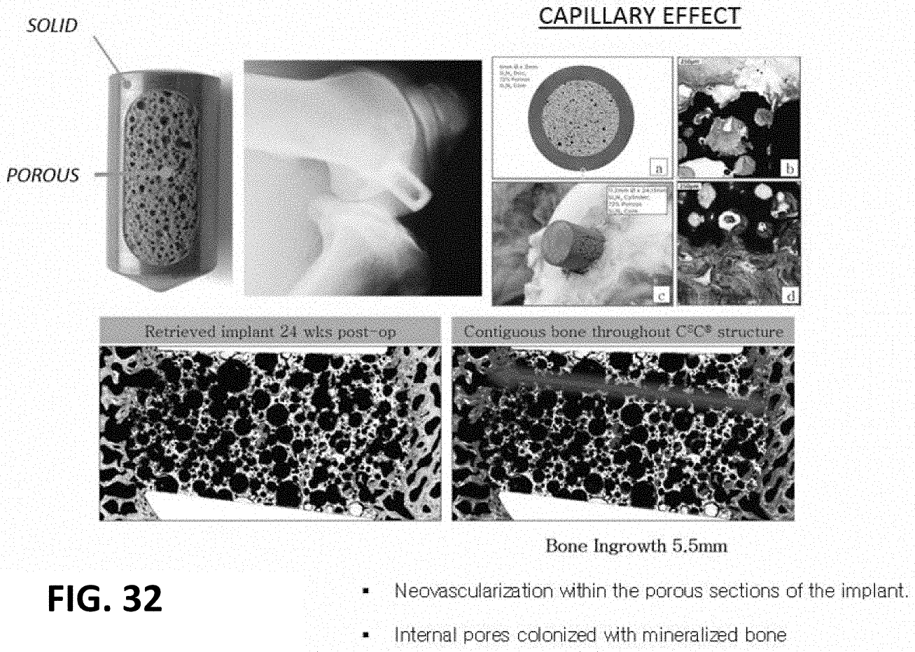

FIG. 30 depicts a variety of exemplary surface and/or subsurface feature treatments and/or feature densities that can be incorporated into a given implant design. As best seen in FIG. 31, such surface and/or subsurface features can be integrated into the implant using a variety of existing manufacturing techniques. Desirably, many of the surface features can include subsurface components, such as internal porous implant sections which seek to induce a capillary-like effect on body cells involved in the process of ossification (see FIG. 32).

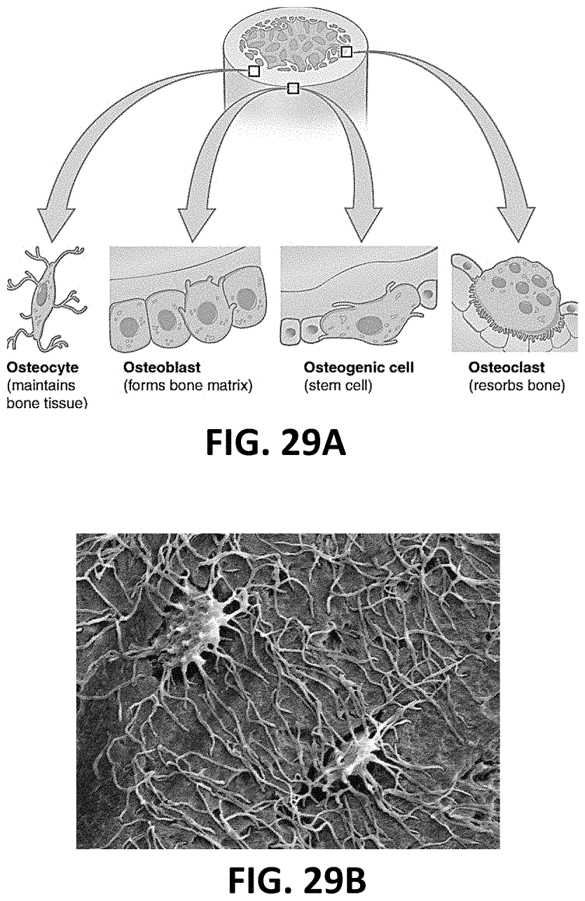

In various embodiments, spinal cages and/or other implants can be created that incorporate bone growth enhancing features such as surface texturing, through holes and/or bone adhesion surfaces such as those described herein. The human body is in a constant state of bone remodeling, which is a process which maintains bone strength and ion homeostasis by replacing discrete parts of old bone with newly synthesized packets of proteinaceous matrix. Bone is resorbed by osteoclasts and is deposited by osteoblasts in a process called ossification (see FIGS. 29A and 29B). Osteocyte activity plays a key role in this process. Desirably, if a porosity (i.e., macro-hole dimension) of a given surface is at least 10× or greater in magnitude than the size of a bone cell, the bone cell should better grow through the pore. Moreover, if the surface textures (i.e., micro-surface irregularities) are near to or approximate the size of a bone cell and/or its features, the bone cell should better attach to the surface. In various embodiments, one objective of the disclosed invention can include the creation of a porous surface and/or texture design suitable for use in 3D printing pf implants as well as the use of machinable porosities, machinable textures and/or laser texturing of implant surfaces and/or other features as an improvement upon pre-existing manufacturing methods for implants. Such techniques can include (1) improvement in osteoconductivity, osteoinductivity and/or anti-bacterial effects of PEEK or titanium implant materials, (2) the use of “drilled through” textured openings using drilling and/or machining operations, and/or (3) the creation of “Peaks and Valleys” surface texturing on implants using laser machining and/or ablation.

Various of the disclosed improvements can be utilized in conjunction with virtually any existing and/or pre-existing implant or cage design, including the employment of rough textured zones in some areas of an implant and the creation of full or partial depth holes in other areas of an implant. The improvements described herein include incorporation into some or all of the Matisse ACIF cages, the MONET ACIF cages, the MONDRIAN LIF cages, and/or other implants disclosed at “https://www.ctlamedica.com/”, including any devices usable for ALIF, DLIF, PLIF and/or TLIF procedures, commercially available from CTL Medical Corporation of Addison, TX, USA (which web disclosures are incorporated herein by reference in their entireties).

In accordance with another aspect of the present subject matter, various methods for manufacturing implants, including silicon nitride implants, cements and/or components thereof, as set for within any of the details described with the present application, are provided.

While embodiments and applications of the present subject matter have been shown and described, it would be apparent that other embodiments, applications and aspects are possible and are thus contemplated and are within the scope of this application.

As previously noted, the various bone cement formulations, bone implants and/or components thereof disclosed herein can incorporate a silicon nitride material (i.e., Si₃N₄ and/or chemical analogues thereof) in their construction, either in the entirety of the implant as well as components, portions, layers, fillings and/or surfaces thereof. The incorporation of silicon nitride as a component material for spinal or other implants can provide significant improvements over existing implant materials and material designs currently available, as the silicon nitride material(s) will desirably be highly osteo-inductive and/or osteoconductive and will facilitate and/or promote implant fixation to adjacent living bone surfaces, while concurrently reducing and/or inhibiting periprosthetic infection and/or bacterial adhesion to the surfaces and/or interior portions of the implant. In various embodiments, materials including silicon nitride materials of differing compositions and/or states (i.e., solid, liquid and/or flowable or moldable “slurry” states, for example) could be utilized in a single implant and/or portions thereof, including the use of solid silicon nitride for an arthroplasty cage implant, with a moldable silicon nitride “paste” placed within a centrally positioned “graft chamber” of the implant. If desired, an implant could include some portion or insert formed from a silicon nitride cement, wherein the silicon nitride or similar component could extend completely through an implant, or only extend partially into and/or out of an implant. For example, FIG. 9 depicts various cross-sectional views of spinal implants with various exemplary silicon nitride insert geometries formed therein, which can include the introduction of such silicon nitride materials in an uncured form which can then cure in situ, if desired.

In various embodiments the disclosed implants may incorporate materials such as silicon nitride that are “phase stable” to a desired degree. For example, various embodiments may desirably withstand standard autoclave sterilization conditions such as 120° C. 1 atmosphere steam for up to 100 hours of time, with no appreciable change in phase composition, no appreciable change in flexural strength and an inherently stable microstructure. Moreover, such materials could desirably provide favorable imaging characteristics, such as high levels of radiolucency and/or no significant MRI or CT scan artifacts.

In various embodiments, a surgical tool kit could include an implant and one or more modular components for the system, including individual silicon nitride components or a modular replacement, if desired. The various components of these systems could optionally be provided in kit form, with a medical practitioner having the option to select an appropriately sized and/or shaped implant and/or modular components to address a desired surgical situation.

Note that, in various alternative embodiments, variations in the position and/or relationships between the various figures and/or modular components are contemplated, such that different relative positions of the various modules and/or component parts, depending upon specific module design and/or interchangeability, may be possible. In other words, different relative adjustment positions of the various components may be accomplished via adjustment in separation and/or surface angulation of one of more of the components to achieve a variety of resulting implant configurations, shapes and/or sizes, thereby accommodating virtually any expected anatomical variation.

Of course, method(s) for manufacturing the various cement formulations, silicon nitride components and/or surgical devices and related components and implanting an implant device into a spine are contemplated and are part of the scope of the present application.

While embodiments and applications of the present subject matter have been shown and described, it would be apparent to those skilled in the art that many more modifications are possible without departing from the inventive concepts herein. The subject matter, therefore, is not to be restricted except in the spirit of the appended claims.

All references, including publications, patent applications, and patents, cited herein are hereby incorporated by reference to the same extent as if each reference were individually and specifically indicated to be incorporated by reference and were set forth in its entirety herein.

The various headings and titles used herein are for the convenience of the reader and should not be construed to limit or constrain any of the features or disclosures thereunder to a specific embodiment or embodiments. It should be understood that various exemplary embodiments could incorporate numerous combinations of the various advantages and/or features described, all manner of combinations of which are contemplated and expressly incorporated hereunder.

The use of the terms “a” and “an” and “the” and similar referents in the context of describing the invention are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., i.e., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventor for carrying out the invention. Variations of those preferred embodiments may become apparent to those of ordinary skill in the art upon reading the foregoing description. The inventor expects skilled artisans to employ such variations as appropriate, and the inventor intends for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A hybrid bone cement comprising a mixture of a bone cement or a bone cement precursor and a plurality of silicon nitride granules, the plurality of silicon nitride granules having an average diameter of 10 μm to 1.5 mm.

2. The hybrid bone cement of claim 1, wherein the plurality of silicon nitride granules comprises at least two different preselected sizes, or range of sizes, of silicon nitride granules.

3. The hybrid bone cement of claim 2, wherein the two different preselected sizes of silicon nitride granules comprise a larger granule size of 500 μm to 1.5 mm and a smaller granule size of 10 μm to 500 μm.

4. The hybrid bone cement of claim 1, wherein the silicon nitride granules comprise a plurality of substantially spherical granules.

5. The hybrid bone cement of claim 1, wherein the bone cement, or the bone cement precursor on conversion to bone cement comprises PMMA, PAA, a calcium phosphate, or calcium sulphate.

6. The hybrid bone cement of claim 1, further comprising one or more biologically or pharmaceutically active compounds.

7. The hybrid bone cement of claim 6, wherein the pharmaceutically active compound is a cell growth factor or bone morphogenic protein.

8. The hybrid bone cement of claim 1, wherein the bone cement comprises a two-part mixture of powdered PMMA and a liquid monomer.

9. The hybrid bone cement of claim 8, wherein the silicon nitride granules are mixed with a dispersing agent prior to the powdered PMMA and liquid monomer being mixed together.

10. The hybrid bone cement of claim 8, wherein a weight ratio of the bone cement to the silicon nitride granules is approximately 1:1.

11. The hybrid bone cement of claim 8, wherein a weight ratio of the bone cement to the silicon nitride granules is at least 10:1 or greater.

12. The hybrid bone cement of claim 8, wherein a weight ratio of the bone cement to the silicon nitride granules is at least 1:10 or greater.

13. The hybrid bone cement of claim 8, wherein a volume ratio of the bone cement to the silicon nitride granules is approximately 1:1.

14. The hybrid bone cement of claim 8, wherein a volume ratio of the bone cement to the silicon nitride granules is at least 10:1 or greater.

15. The hybrid bone cement of claim 8, wherein a volume ratio of the bone cement to the silicon nitride granules is at least 1:10 or greater.

16. A hybrid bone cement comprising a mixture of a PMMA bone cement or a PMMA bone cement precursor which hardens to a substantially solidified material and a plurality of particles of a silicon nitride powder.

17. The hybrid bone cement of claim 16, wherein the plurality of silicon nitride particles comprise an average diameter of 10 μm to 1.5 mm.

18. The hybrid bone cement of claim 16, wherein at least a portion of the plurality of silicon nitride particles comprise an average diameter of 10 μm to 1.5 mm.

19. The hybrid bone cement of claim 16, wherein the plurality of silicon nitride particles comprise an average diameter of less than 0.1 mm.

20. The hybrid bone cement of claim 16, wherein a volume ratio of the PMMA bone cement or PMMA bone cement precursor to the silicon nitride granules is at least 10:1 or greater.
